(19) United States
(12) Patent Application Publication
(10) Pub. No.: US 2024/0191301 A1
(43) Pub. Date: Jun. 13, 2024

(54) Methods, Systems, and Compositions for Diagnosing Transplant Rejection

(71) Applicants: Northwestern University, Evanston, IL (US); Transplant Genomics, Inc., Framingham, MA (US); The Scripps Research Institute, La Jolla, CA (US)

(72) Inventors: John Friedewald, Chicago, IL (US); Michael M. Abecassis, Highland Park, IL (US); Rohita Sinha, Lee's Summit, MO (US); Sook Hyeon Park, Evanston, IL (US); Lihui Zhao, Evanston, IL (US); Juston Weems, Lenexa, KS (US); Sunil M. Kurian, San Diego, CA (US); Steve Kleiboeker, Lenexa, KS (US)

(73) Assignees: Northwestern University, Evanston, IL (US); Transplant Genomics, Inc., Framingham, MA (US); The Scripps Research Institute, La Jolla, CA (US)

(21) Appl. No.: 18/423,804

(22) Filed: Jan. 26, 2024

Related U.S. Application Data
(63) Continuation of application No. PCT/US22/38728, filed on Jul. 28, 2022.
(60) Provisional application No. 63/227,276, filed on Jul. 29, 2021.

(51) Int. Cl. C12Q 1/6883

(52) U.S. Cl. CPC C12Q 1/6883; C12Q 2600/112; C12Q 2600/118; C12Q 2600/158

(57) ABSTRACT

Described herein are methods, compositions, and systems useful for detecting transplant rejection and associated abnormal conditions in solid organ transplant recipients, such as kidney transplant recipients. Methods described herein may involve combined assessment of blood gene expression profiles from an assessment of particular, related mRNA transcript levels and donor-derived cell-free nucleic acids (dd-cfDNA).

### CROSS-REFERENCE TO RELATED APPLICATIONS

This application is a continuation of International Patent Application No. PCT/US2022/038728, filed Jul. 28, 2022, which claims the benefit of priority to U.S. Provisional Patent Application No. 63/227,276, filed Jul. 29, 2021, which is incorporated in its entirety herein by reference.

### FIELD

Described herein are methods, compositions, and systems useful for detecting transplant rejection and associated abnormal conditions in solid organ transplant recipients, such as kidney transplant recipients. Methods described herein may involve combined assessment of blood gene expression profiles from an assessment of particular, related mRNA transcript levels and donor-derived cell-free nucleic acids (dd-cfDNA).

### BACKGROUND

Rejection in a solid organ transplant recipient, such as a kidney transplant recipient, can manifest as clinical acute rejection, detectable by phenotypic markers such as serum creatinine levels for a kidney transplant recipient, or a subclinical acute rejection, for example, which may not be detectable with commonly used clinical markers. Subclinical acute rejection, for example, is associated with worse clinical outcomes, including higher risk of subsequent clinical acute rejection, de novo donor-specific antibody (DSA) formation and associated antibody-mediated rejection, and graft fibrosis. Several clinical trials suggest that treating subclinical rejection improves outcomes. Monitoring patients for subclinical rejection typically involves serial surveillance biopsies to detect the rejection. However, despite clinical evidence, only about half of high-volume transplant programs in the United States perform surveillance biopsies. In addition, surveillance biopsies are expensive, painful, and risky for patients (with complications that can include graft loss), and only lead to about 15-25% positivity, indicating that as many as 85% of surveillance biopsies are not necessary. (First et al., Transplantation Proceedings 51: 729-33 (2019).) Hence, noninvasive methods of assessing the status of a solid organ transplant are needed.

In addition, rejection may be T cell mediated (cellular mediated rejection) or it may be antibody-mediated, or a combination of the two, which may lead to different treatments, depending on which is detected. Improved screening of both clinical and subclinical acute rejection in solid organ transplant recipients may also assist in detecting the primary cause of the rejection—cellular mediated or antibody mediated or both, which may assist in determining the best treatments in response to the rejection.

### SUMMARY

As indicated above, there is a need for improved methods, systems, and compositions for detecting rejection in solid organ transplant recipients as an alternative to surveillance biopsies. The present disclosure relates to methods for distinguishing rejection from non-rejection in solid organ transplant recipients, in some cases those showing no clinical symptoms of rejection, and in other cases in those showing clinical signs of rejection. Methods herein include determining both the level of donor-derived, cell-free DNA (dd-cfDNA) and the expression level of at least one mRNA transcript in a sample from a solid organ transplant recipient, such as a blood or plasma or serum sample. In some cases, the recipient does not show clinical signs of rejection. In some cases, the methods help to distinguish cellular mediated rejection from antibody mediated rejection in that the level of dd-cfDNA and the expression level of the at least one mRNA transcript tend to correlate more with one of these two types of rejection over the other, thus providing a more precise determination of the rejection status of a recipient. The present disclosure also relates to methods of distinguishing rejection from non-rejection in a subject that shows signs of clinical rejection, such as a high serum creatinine level in a kidney transplant subject, by determining the level of dd-cfDNA. The present disclosure also relates to methods of distinguishing rejection from non-rejection in a subject that does not show signs of clinical rejection, such as with a serum creatinine level of less than 2.3 mg/dL, in a kidney transplant subject, by determining the level of dd-cfDNA.

Some exemplary methods herein include, for example, methods of distinguishing rejection from non-rejection in a kidney transplant recipient, comprising (a) obtaining a blood, plasma, or serum sample from the kidney transplant recipient: (b) obtaining cell-free DNA (cfDNA) and mRNA from the sample: (c) determining (i) the level of donor derived cell-free DNA (dd-cfDNA) in the cfDNA and (ii) the expression level of at least one mRNA transcript, wherein the at least one mRNA transcript shows significantly different expression levels in kidney transplant rejection compared to kidney transplant non-rejection subjects; and (d) distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) result of a trained algorithm based on the expression level of the at least one mRNA transcript indicating rejection or non-rejection, wherein the algorithm compares the expression profile of the at least one mRNA transcript of the recipient to the expression profile of kidney transplant subjects with and without rejection. In some cases, rejection in the recipient is indicated by a pre-determined threshold value of dd-cfDNA of ≥ 0.5%, ≥ 0.6%, ≥ 0.7%, ≥ 0.8%, ≥ 0.9%, ≥ 1%, ≥ 1.2%, ≥ 1.5%, or ≥ 2%. In some cases, rejection in the recipient is indicated by a pre-determined threshold value of dd-cfDNA of ≥ 0.7%, optionally wherein determining the dd-cfDNA level utilizes data from recipient genotype information. In some cases, the methods comprise determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample. In some cases, the at least one mRNA transcript comprises one or more of the mRNA transcripts of Table A, such as 2-120, 5-120, 10-120, 50-120, 80-120, 5-50, 10-50, 50-100, or all of the mRNA transcripts of Table A. In some cases, the recipient has a serum creatinine level of <2.3 mg/dL, or an increase of serum creatinine compared to baseline of no more than 10% or no more than 20%. In some cases, the recipient has a serum creatinine level of 2.3 mg/dL or higher, or an increase of serum creatinine compared to baseline of no more than 10% or no more than 20%. In some cases, a method herein is performed at least one month, at least two months, at least three months, at least six months, or at least one year after transplantation. In some cases, the expression level of the at least one mRNA transcript is determined by reverse transcription PCR (RT-PCR) (such as quantitative RT-PCR), hybridization to an array, or next generation sequencing. In some cases, the dd-cfDNA level is determined by whole genome sequencing. In some cases, determining the dd-cfDNA level comprises comparison of recipient and donor genotype information, and in other cases the dd-cfDNA is determined without comparison to donor genotype information. In some cases, the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects. In some cases, the method is capable of further distinguishing likelihood of acute cellular rejection from antibody-mediated rejection, wherein the dd-cfDNA level indicates presence or absence of antibody-mediated rejection, and wherein the level of the at least one mRNA transcript indicates presence or absence of acute cellular rejection. In some cases, the method has a negative predictive value (NPV) of at least 85%, at least 87%, at least 88%, at least 90%, at least 92%, or at least 94% when both the level of dd-cfDNA is below the pre-determined threshold value and the result of a trained algorithm based on the expression level of the at least one mRNA transcript does not indicate rejection. In some cases, the method has a positive predictive value (NPV) of at least 80%, at least 81%, at least 82%, at least 84%, at least 86%, at least 88%, or at least 89% when both the level of dd-cfDNA is at or above the pre-determined threshold value and the result of a trained algorithm based on the expression level of the at least one mRNA transcript indicates rejection. In some cases, determining the dd-cfDNA level utilizes data from recipient genotype information and wherein the expression level of the at least one mRNA transcript is determined by reverse-transcription PCR (RT-PCR) (such as quantitative RT-PCR). In some cases, the pre-determined threshold value of the dd-cfDNA is determined by a multivariate regression algorithm that comprises dd-cfDNA levels and expression levels of the at least one mRNA transcript in a set of transplant recipients who received the same solid organ transplant as the recipient.

All publications, patents, and patent applications cited in this disclosure (either in the text or in a reference list) are incorporated by reference herein in their entireties. Further description of embodiments of the disclosure is provided in the sections that follow and in the drawings.

### BRIEF DESCRIPTION OF THE DRAWINGS

A better understanding of certain features and advantages of the embodiments described herein may be obtained by reference to the accompanying drawings, summarized below:

FIG. 1 (FIG. 1) depicts a CONSORT (Consolidated Standards of Reporting Trials) diagram illustrating the number of patients and then samples available for analysis based on inclusion and exclusion criteria in addition to sample availability for a clinical study described in Example 1.

FIGS. 2A-2D (FIG. 2A-2D) depict Area under Receiver Operating Curve (AUROC) analyses (as graphs) for gene expression profile and dd-cfDNA assays, by rejection type vs. No Rejection. FIG. 2A depicts the analysis of gene expression profile only for distinguishing acute cellular rejection vs. no rejection. FIG. 2B depicts the analysis of donor derived cfDNA only for distinguishing acute cellular rejection vs. no rejection. FIG. 2C depicts the gene expression profile only for antibody mediated rejection vs. no rejection. FIG. 2D shows donor derived cfDNA only for antibody mediated rejection vs. no rejection.

FIG. 3A (FIG. 3A) depicts an overall summary of gene expression profile and donor derived cfDNA assays by sample type as a diagram. FIG. 3A depicts all cases of subclinical rejection vs no rejection by biopsy and assay result. For FIG. 3A, of 428 samples, the subclinical rejection group (n=103: dotted, in which rejection was revealed upon biopsy) consists of gene expression profile alone positive (n=23: hatched), donor derived cfDNA alone positive (n=27; diagonal lines), both gene expression profile and donor derived cfDNA positive (n=21), and both gene expression profile and donor derived cfDNA negative (n=32). Of the normal biopsies (n=325: plain white), both tests were negative (n=242), both positive (n=5), gene expression profile alone positive (n=45: hatched), and donor derived cfDNA alone positive (n=33: diagonal lines).

FIG. 3B (FIG. 3B) shows data for all cases of subclinical rejection broken down by rejection type as well as assay result, acute cellular rejection and antibody mediated rejection. FIG. 3B shows that the 103 subclinical rejection cases are divided by histology phenotypes into antibody mediated rejection alone (n=42: right side of figure), acute cellular rejection alone (n=38: left side of figure), and combined acute cellular rejection and antibody mediated rejection (n=23: bottom of figure). The breakdown of acute cellular rejection by Banff grade is also shown. The numbers in both FIGS. 3A and 3B demonstrate the true positives and false negatives found in each assay. Although there is some overlap, the two assays tended to detect different types of rejection. In FIG. 3B, 1A=Banff 1A acute cellular rejection, 1B=Banff 1B acute cellular rejection, 2A=Banff 2A acute cellular rejection, and BL=borderline cellular rejection.

FIGS. 4A-4D (FIG. 4A-4D) depict performance metrics of individual gene expression profile and donor derived cfDNA assays compared with the logistic regression model with continuous variables for combined gene expression profile and donor derived cfDNA to distinguish subclinical rejection vs. no rejection. FIG. 4A depicts AUROC of gene expression profile only for subclinical rejection vs. no rejection. FIG. 4B depicts AUROC of combined gene expression profile and donor derived cfDNA performance on the Clinical Trials in Organ Transplant 08 (CTOT 08) cohort (Training Set) by multivariable logistic regression model using the continuous score output of both tests. FIG. 4C depicts AUROC of donor derived cfDNA only for subclinical rejection vs. no rejection. FIG. 4D depicts AUROC of an external validation with an independent cohort (n=105 samples) by multivariable logistic regression model using the continuous score output of both tests.

FIG. 5 (FIG. 5) depicts a distribution of samples by clinical phenotype, gene expression profile probability score, and % donor derived-cfDNA as described in Example 1. Shown is a scatterplot of samples based on their clinical phenotype, Negative, i.e., TX=no rejection, and Positive, i.e., Subclinical Acute Rejection (subAR)), TruGraf® Gene Expression Profile (GEP) probability score (which is scaled on a 0-1 scale with scores >0.50 being positive (i.e., indicating rejection) and scores <0.50 being negative, i.e., indicating no rejection), and % donor-derived cell free DNA (dd-cfDNA) with a cutoff of 0.7% for the Viracor TRAC® assay.

FIG. 6 (FIG. 6) depicts a schematic showing how patient samples can be classified according to methods described herein.

FIG. 7 (FIG. 7) depicts an example computer system for executing methods according to the disclosure.

FIG. 8 (FIG. 8) shows an example data processing pipeline of one potential method of cfDNA sequencing and genotyping as described herein, which relies on genotype data from a recipient. Illustration of the pipeline used to retrieve allele counts in cfDNA fragments for each recipient-genotyped SNP from the raw cfDNA sequencing and genotyping measurements is shown.

### DETAILED DESCRIPTION

### Definitions

Unless defined otherwise, all technical and scientific terms used herein have the same meaning as commonly understood by those of ordinary skill in the art to which this invention pertains. In addition, the following definitions are provided to assist the reader in the practice of the invention.

The term “or” as used herein and throughout the disclosure is intended as an inclusive “or,” meaning “and/or” unless the context expressly indicates otherwise.

The terms “a” or “the” as used herein and throughout the disclosure are intended to encompass both singular and plural, i.e., to mean “at least one,” unless the context expressly indicates otherwise.

The terms “transplantation” or a “transplant” generally refer to the transfer of tissues, cells, or a solid organ from a donor individual into a recipient individual. A donor and recipient may or may not be from the same species. Thus, for example, a human recipient may receive a solid organ from a non-human animal in some embodiments. An “allograft” further indicates a transfer of tissues, cells, or a solid organ between different individuals of the same species. In contrast, if the donor and recipient are the same individual, the graft is referred to as an “autograft.”

A “recipient” generally refers to an individual receiving a transplant, allograft, or autograft. A “recipient” herein is a human, unless expressly stated otherwise (i.e., a murine recipient or the like). The terms “individual,” “subject,” or “patient” in the context of transplantation or medical treatment generally refer interchangeably to a human receiving such a transplantation or other medical treatment, e.g., a recipient of a transplant or of other medical treatment.

As used herein, a recipient that does not have rejection, or that shows “non-rejection,” or is negative for rejection, or the like, which may also be abbreviated “TX” herein, generally signifies that the recipient does not exhibit symptoms or test results indicating organ dysfunction or rejection. Accordingly, in such recipients the transplant is considered a normal functioning transplant. A TX patient can have normal histology on a surveillance biopsy (e.g. no evidence of rejection), and in the context of a kidney transplant recipient: Banff i=0 with t=0 or 1, g=0, ptc=0, interstitial fibrosis=0 or 1, tubular atrophy=0 or 1 and stable renal function (e.g. serum creatinine <2.3 mg/dl and/or <20% increase in creatinine compared to a minimum of 2-3 prior values).

In contrast, a “rejection” (also termed “non-TX” herein) can be observed either clinically or subclinically, for example, such as via biomarker tests herein or via histology. For example, a clinical rejection in the case of a kidney transplant recipient may be indicated by a serum creatinine at or above 2.3 mg/dl and/or an increase in creatinine of 20% or more compared to a minimum of 2-3 prior values. The term “rejection” herein encompasses several sub-types of rejection, such as clinical or subclinical acute rejection, acute cellular rejection, and antibody-mediated rejection.

“Acute rejection (AR)” or “clinical acute rejection” generally refers to a condition that can occur when transplanted tissue is rejected by the recipient's immune system, which damages or destroys the transplanted tissue unless immunosuppression is achieved. T-cells, B-cells and other immune cells as well as possibly antibodies of the recipient may cause the graft cells to lyse or produce cytokines that recruit other inflammatory cells, eventually causing necrosis of allograft tissue. In some instances, AR can be diagnosed by a biopsy of the transplanted organ. In the case of kidney transplant recipients, AR can be associated with an increase in serum creatinine levels. AR can occur more frequently in the first three to 12 months after transplantation but there is a continued risk and incidence of AR for the first five years post-transplant and whenever a patient's immunosuppression becomes inadequate for any reason for the life of the transplant.

As used herein, the term “subclinical acute rejection” (also “subAR”) or “subclinical rejection” refers to histologically defined acute rejection—including but not limited to histologically defined acute cellular rejection—characterized by tubule-interstitial mononuclear infiltration identified from a biopsy specimen (e.g. histology on a surveillance biopsy consistent with acute rejection such as ≥Banff borderline cellular rejection and/or antibody mediated rejection), but without the requirement of functional deterioration. In some instances, subAR can represent the beginning or conclusion of an alloimmune infiltrate diagnosed fortuitously by protocol sampling, and some episodes of clinical rejection may actually represent subAR with an alternative cause of functional decline, such as concurrent calcineurin inhibitor (CNI) nephrotoxicity. A subAR subject can have normal and stable organ function. SubAR can be distinguished from acute rejection, as acute rejection requires acute renal impairment. The differences between subAR and acute rejection can involve real quantitative differences of renal cortex affected, qualitative differences (such as increased perforin, granzyme, c-Bet expression or macrophage markers), or an increased ability of the allograft to withstand immune injury (‘accommodation’). SubAR is often diagnosed only on biopsies taken as per protocol at a fixed time after transplantation, rather than driven by clinical indication, and is accordingly difficult to detect by traditional kidney function measurements like serum creatinine and glomerular filtration rates.

Subclinical acute rejection may comprise either or both of “acute cellular rejection,” which may also be called “T cell mediated rejection” or “cell mediated rejection,” and “antibody-mediated rejection.” T cell mediated rejection, for example, may be associated with an increase in activity of certain T cell populations in the vicinity of the transplanted organ or tissue, or markers for such cells. Antibody-mediated rejection, for example, may be associated with injury to the transplanted tissue or organ, and may be characterized by the production of IgG antibodies against the transplanted tissue, such as anti-HLA antibodies.

A “likelihood” of a particular type of subclinical rejection may be obtained in methods herein. For example, certain biomarker tests, when positive, tend to correlate with a particular type of subclinical rejection such as antibody-mediated rejection or acute cellular rejection over another type of rejection, thus indicating that the subject is likely to have a particular type of rejection over another.

As used herein, in performing the methods, “obtaining a sample” includes obtaining a sample directly or indirectly. In some embodiments, the sample is taken from the subject by the same party (e.g. a testing laboratory) that subsequently acquires biomarker data from the sample. In some embodiments, the sample is received (e.g. by a testing laboratory) from another entity that collected it from the subject (e.g. a physician, nurse, phlebotomist, or medical caregiver). In some embodiments, the sample is taken from the subject by a medical professional under direction of a separate entity (e.g. a testing laboratory) and subsequently provided to said entity (e.g. the testing laboratory). In some embodiments, the sample is taken by the subject or the subject's caregiver at home and subsequently provided to the party that acquires biomarker data from the sample (e.g. a testing laboratory). As used herein, when a method herein is said to be conducted at a particular time, such as a specific time after transplantation (e.g., 1 week, 1 month, etc. following transplantation), where there is a delay between the time that the sample was taken from the recipient and when the dd-cfDNA and mRNA transcript expression data were obtained, the method is said to be conducted at the time that the sample was taken from the recipient, since the results reflect the state of the recipient at that point in time.

As used in methods herein, the term “mRNA transcript” indicates an mRNA obtained from transcription of a particular gene, and includes full length and non-full length transcripts and transcripts that result from alternative splicing. Thus, each “mRNA transcript” herein is from a different gene, and a reference to two or more mRNA transcripts, or, for example to 50 or 100 mRNA transcripts, herein means the mRNA transcripts of two or more genes or of 50 or 100 genes. An “mRNA transcript” is not necessarily a single RNA molecule. For example, due to degradation of RNA in a recipient sample, an original mRNA transcript for a gene may be degraded into multiple RNA molecules that cover the length of the transcribed coding region. But an “mRNA transcript” includes sufficient transcription of the gene coding region to be uniquely identified as belonging to the particular, transcribed gene, and thus, to be a marker of the level of expression of that gene.

The term “significantly different” in the methods herein, i.e. in referring to genes whose mRNA transcripts show changes in expression levels in rejection vs. non-rejection subjects, means statistically significantly different, such as through a T-test and an associated P value that indicates statistical significance. Similarly, if other mRNA transcripts show changes in expression levels that are “not significantly different.” the changes are not statistically significantly different.

A “biopsy” generally refers to a specimen obtained from a living patient for diagnostic or prognostic evaluation. A “surveillance biopsy” for example may be performed following a transplant to look for evidence of rejection or non-rejection.

The term “treatment.” for example, for a transplant recipient, includes medical management strategies such as active surveillance, which may include diagnostic or biopsy assays to assess likelihood of rejection, as well as therapeutic treatment, for example, with drugs intended to suppress rejection or promote functioning of the transplanted organ, such as immunosuppressants. Further discussion of treatments is provided below.

Additional definitions of particular terms are provided in the sections that follow.

Methods of Distinguishing Rejection from Non-Rejection

The present disclosure relates to methods capable of distinguishing rejection from non-rejection in a solid organ transplant recipient that, in some embodiments, combine determination of the level of donor-derived, cell free DNA (dd-cfDNA) in a sample from the recipient with determining the expression level of at least one mRNA transcript in the sample, and analyzing results of both assays. Certain methods herein comprise: obtaining a sample from the solid organ transplant recipient: obtaining cell-free DNA (cfDNA) and mRNA from the sample: determining (i) the level of donor derived cell-free DNA (dd-cfDNA) in the cfDNA and (ii) the expression level of at least one mRNA transcript, wherein the at least one mRNA transcript shows significantly different expression levels in kidney transplant rejection compared to kidney transplant non-rejection subjects: and distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) expression level of the at least one mRNA transcript or a result of an algorithm based on the expression level indicating rejection.

### Exemplary Samples

The methods in some embodiments may be conducted on a single sample from the recipient, for instance, a blood, serum, plasma, urine, or tissue sample, or a sample obtained by a non-invasive, minimally-invasive, or invasive procedure as discussed below. In some embodiments, the dd-cfDNA and mRNA transcript information are obtained from a single sample from the recipient. Such a “single sample” means herein a sample that is obtained from the recipient at one time, such as during one blood draw or phlebotomy appointment or during one other diagnostic or medical appointment. Accordingly, the “single sample” is not required to be present in the same sample container, but instead is merely drawn from the patient at the same time, during the context of one diagnostic or medical appointment. In other cases, the dd-cfDNA and mRNA transcript information are obtained on different samples from the subject, such as obtained at roughly the same time, but of different types (e.g., blood draw and a tissue sample).

In some embodiments, the sample is obtained from a non-invasive procedure, such as a throat swab, buccal swab, bronchial lavage, urine collection, skin or epidermal scraping, feces collection, menses collection, or semen collection. In other cases, a minimally-invasive procedure may be used such as a blood draw, e.g., by venipucture methods. In other cases, a sample may be obtained by an invasive procedure such as a biopsy, alveolar or pulmonary lavage, or needle aspiration.

In some embodiments, the sample is a blood, serum, or plasma sample. A “blood” sample, herein refers to whole blood or fractions thereof, including plasma, lymphocytes, peripheral blood lymphocytes (PBLs), peripheral blood mononuclear cells (PBMCs), serum, T cells. B Cells, CD3 cells, CD8 cells, CD4 cells, or other immune cells. In some embodiments, it is a whole blood sample. Other samples that can be analyzed include urine, feces, saliva, and tissue from a biopsy. However, a sample may be any material containing tissues, cells, nucleic acids, genes, gene fragments, expression products, polypeptides, exosomes, gene expression products, or gene expression product fragments of a transplant recipient to be tested.

In some embodiments, a whole blood sample drawn from the recipient for analysis according to the methods herein may be, for example, 10 mL or less, 8 mL or less, 7 mL or less, 6 mL or less, or 5 mL or less. In some embodiments, a blood sample may be 6 mL or less. A blood sample may be obtained by a minimally-invasive method such as a blood draw or fingerstick or dried blood spot (DBS). The sample may be obtained by venipuncture or fingerstick via lancet device. Some or all of a sample obtained from a recipient may then be used in the methods. In some embodiments, multiple samples may be obtained by the methods herein to ensure a sufficient amount of biological material. In some cases, methods herein may be performed on more than one recipient's sample, i.e., on pooled samples, then deconvoluted to determine whether any of the samples indicate rejection.

### Exemplary Solid Organ Transplant Recipients

A solid organ transplant recipient may be a recipient of a solid organ or a fragment of a solid organ such as a kidney, heart, liver, pancreas, or lung. In some embodiments, the transplant recipient is a kidney transplant recipient, for example, who has undergone a kidney transplantation medical procedure. Recipients herein are humans unless specifically stated to be a different animal, such as a non-human primate (e.g., ape, monkey, chimpanzee), a domestic animal such as a cat, dog, or rabbit, or a livestock animal such as a goat, horse, cow, pig, or sheep, or a laboratory animal such as a rodent, mouse, SCID mouse, rat, guinea pig, etc.

The donor organ, tissue, or cells may be derived from a subject who has certain similarities or compatibilities with the recipient subject. For example, the donor organ, tissue, or cells may be derived from a donor subject who is age-matched, ethnicity-matched, gender-matched, blood-type compatible, or HLA-type compatible with the recipient subject. In some circumstances, the donor organ, tissue, or cells may be derived from a donor subject that has one or more mismatches in age, ethnicity, gender, blood-type, or HLA markers with the transplant recipient due to organ availability. The organ may be derived from a living or deceased donor.

In various embodiments, recipients have undergone an organ transplant within 6 hours, 12 hours, 1 day, 2 days, 3 days. 4 days, 5 days. 10 days, 15 days, 20 days, 25 days, 1 month, 2 months, 3 months, 4 months, 5 months, 7 months, 9 months, 11 months, 1 year, 2 years, 4 years, 5 years, 10 years, 15 years, 20 years or longer of prior to being assessed by a method herein.

In some embodiments, the recipient is undergoing a treatment regimen, or being evaluated for a treatment regimen, such as immunosuppressive therapy, to inhibit rejection or to reduce at least one symptom of rejection. However, in some instances, the recipient is not undergoing a treatment regimen such as immunosuppressant therapy. In some embodiments, the subject is receiving a standard of care immunosuppressant therapy regimen for the type of solid organ transplant received. In some embodiments, the recipient has not received a biopsy, such as a surveillance biopsy prior to assessment via a method herein.

In some embodiments, the recipient has received at least one immunosuppressive drug, and, if the result of the method indicates that the recipient has clinical or subclinical acute rejection, the method comprises increasing the frequency or dosage of the at least one immunosuppressant drug, administering a further immunosuppressant drug, or administering a different immunosuppressive drug to the recipient. In some cases, if the method indicates that the recipient has clinical or subclinical acute rejection, following such adjustment of immunosuppressant therapy, the method is repeated to assess the effect of such therapy adjustment, for instance, after 1 week, 2 weeks, 1 month, 2 months, 3 months, 6 months or one year following the adjustment in the therapy. In some cases, if the method indicates that the recipient has clinical or subclinical rejection, a surveillance biopsy is ordered for the recipient, optionally, along with or prior to an adjustment in immunosuppressive therapy, such as increasing the frequency or dosage of the at least one immunosuppressant drug, administering a further immunosuppressant drug, or administering a different immunosuppressive drug to the recipient.

In some cases, methods herein are performed every 1 month, 2 months, 3 months, 6 months, or year following a transplant procedure, for example. In some cases, they are performed every 2 months. In some cases, every 3 months. In some cases, every 6 months. In some cases, the frequency depends on the test results. Thus, for example, in some cases methods herein may be performed with increased frequency if one or both results is positive, for instance, if treatment is subsequently adjusted.

In some embodiments, the recipient may have undergone other biomarker testing prior to conducting a method herein. For example, in the case of a kidney transplant recipient, serum creatinine levels or estimated glomerular filtration rate may have been determined. For example, serum creatinine levels, estimated glomerular filtration rates, or changes in those parameters may be used to provide an indication of the performance of the kidney transplant. Other organ-specific parameters may be used in the case of a pancreatic, liver, lung, or heart transplant, for example, to assess the performance of the transplanted organ. For instance, in the case of a pancreatic transplant, pancreatic enzymes may be assessed: in a heart or lung transplant recipient, hemodynamic parameters may be assessed: in a liver transplant, liver protein levels may be assessed.

In some cases, a transplant recipient assessed in methods herein may have results from parameters such as those above indicating normal organ function, while in other cases, the recipient may have results indicating impairment in organ function or graft failure. For example, in some cases, a recipient may an “acute dysfunction no rejection (ADNR)” phenotype, in which the subject shows symptoms of or biomarkers associated with dysfunction of the transplanted organ, but does not show symptoms or biomarkers associated with rejection. In some cases, a subject, such as a kidney transplant subject, may show evidence of interstitial fibrosis and tubular atrophy (IFTA) or recurrent glomerular disease.

In some embodiments, the recipient is a kidney transplant recipient. In some such cases, the kidney transplant recipient, prior to obtaining a sample for performing a method herein, has a serum creatinine level indicative of non-rejection, such as <2.3 mg/dL. For example, typical reference ranges for serum creatinine are 0.5 to 1.0 mg/dL for women and 0.7 to 1.2 mg/dL for men, though typical kidney transplant patients have serum creatinine concentrations in the 0.8 to 1.5 mg/dL range for women and 1.0 to 1.9 mg/dL range for men. In some instances, a transplant recipient may have a serum creatinine level of at least 0.1 mg/dL. 0.2 mg/dL, 0.3 mg/dL, 0.4 mg/dL, 0.5 mg/dL, 0.6 mg/dL, 0.7 mg/dL 0.8 mg/dL, 0.9 mg/dL. 1.0 mg/dL, 1.1 mg/dL, 1.2 mg/dL, 1.3 mg/dL, 1.4 mg/dL, 1.5 mg/dL. 1.6 mg/dL, 1.7 mg/dL, 1.8 mg/dL. 1.9 mg/dL. 2.0 mg/dL. 2.1 mg/dL. 2.2 mg/dL. 2.3 mg/dL. 2.4 mg/dL, 2.5 mg/dL. 2.6 mg/dL. 2.7 mg/dL. 2.8 mg/dL. 2.9 mg/dL. 3.0 mg/dL. 3.1 mg/dL. 3.2 mg/dL, 3.3 mg/dL. 3.4 mg/dL. 3.5 mg/dL. 3.6 mg/dL. 3.7 mg/dL. 3.8 mg/dL. 3.9 mg/dL, or 4.0 mg/dL. In some instances, a threshold of <2.3 mg/dL is used to indicate clinical rejection (at or above 2.3 mg/dL) or lack of clinical rejection (<2.3 mg/dL). In some instances, a transplant recipient may have a serum creatinine level of less than 0.5 mg/dl, 0.7 mg/dL. 1.0 mg/dL, 1.1 mg/dL. 1.2 mg/dL. 1.3 mg/dL. 1.4 mg/dL, 1.5 mg/dL. 1.6 mg/dL, 1.7 mg/dL. 1.8 mg/dL, 1.9 mg/dL. 2.0 mg/dL. 2.1 mg/dL. 2.2 mg/dL. 2.3 mg/dL. In some instances, the trend of serum creatinine levels over time can be used to evaluate the recipient's organ function. For example, an increase in serum creatinine level of 10%, 15%, 20%, 25%, 30% or more over a specific time period from a baseline measurement, such as 1-2 weeks post-transplantation, may also be used as a marker for clinical rejection in some cases. In some instances, a transplant recipient may have an increase of a serum creatinine level of at least 10%, 20%, 30%, 40%, 50%, 60%, 70%, 80%, 90%, or 100% from baseline, such as 1-2 weeks post-transplantation. In some cases, the increase in serum creatinine (e.g., any increase in the concentration of serum creatinine described herein) may occur over about 0.25 days, 0.5 days, 0.75 days, 1 day, 1.25 days, 1.5 days, 1.75 days, 2.0 days, 3.0 days, 4.0 days, 5.0 days, 6.0 days, 7.0 days, 8.0 days, 9.0 days, 10.0 days, 15 days, 30 days, 1 month, 2 months, 3 months. 4 months, 5 months, or 6 months, 1 year, 1.5 years, 2 years, or more. In some embodiments, a kidney transplant recipient has an estimated glomerular filtration rate (eGFR) that indicates non-rejection. For example, the transplant recipient may show signs of a transplant dysfunction or rejection as indicated by a decreased eGFR. In some instances, a transplant recipient may have a decrease of a eGFR of at least 10%, 20%, 30%, 40%, 50%, 60%, 70%, 80%, 90%, or 100% from baseline, such as 1-2 weeks post-transplantation. In some cases, the decrease in eGFR may occur over 0.25 days, 0.5 days, 0.75 days, 1 day, 1.25 days, 1.5 days, 1.75 days, 2.0 days, 3.0 days, 4.0 days, 5.0 days, 6.0 days, 7.0 days, 8.0 days, 9.0 days, 10.0 days, 15 days, 30 days, 1 month, 2 months, 3 months, 4 months, 5 months, or 6 months, 1 year, 1.5 years, 2 years, or more. In some instances, methods herein further comprise determining serum creatinine level, eGFR, and/or change in serum creatinine or eGFR. In some cases, a “baseline” against which an increase in serum creatinine levels and/or a decrease in eGFR levels is measured is a time-point post transplantation at which serum creatinine levels are at their lowest, often 1-2 weeks post-transplantation. For example, prior to kidney transplantation, serum creatinine levels may be very high, even with dialysis treatment, such as more than 10 or more than 15 mg/dL, and fall to a nadir after transplantation once the transplanted kidney functions. Changes in serum creatinine and/or eGFR following this low baseline level may then be monitored to assess the continued function of the transplanted kidney.

In some embodiments, a kidney transplant subject has a serum creatinine level and/or eGFR level, or changes in serum creatinine and/or eGFR that indicates non-rejection (i.e., the subject is not found to have clinical rejection). In some instances, a transplant recipient may have a serum creatinine level of less than 0.5 mg/dL, 0.7 mg/dL, 1.0 mg/dL, 1.1 mg/dL, 1.2 mg/dL. 1.3 mg/dL, 1.4 mg/dL, 1.5 mg/dL, 1.6 mg/dL, 1.7 mg/dL, 1.8 mg/dL, 1.9 mg/dL, 2.0 mg/dL. 2.1 mg/dL. 2.2 mg/dL, 2.3 mg/dL. In such cases, methods of the invention can be used to identify hidden subclinical acute rejection, such as acute cellular rejection and/or antibody mediated rejection, without depending on invasive biopsies.

mRNA Expression Profiles

Methods herein, for example, comprise obtaining mRNA from the recipient sample and determining the expression level of at least one mRNA transcript or a result of an algorithm based on the expression level and determining whether the expression level or the algorithm result indicates a likelihood of rejection for the recipient. In some embodiments, the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample. In some cases, the at least one mRNA transcript comprises mRNA transcripts of one or more of the genes provided in Table A below. In some cases, the at least one mRNA transcript comprises 2-120, 5-120, 10-120, 50-120, 80-120, 2-128, 5-128, 10-128, 50-128, 80-128, 5-50, 10-50, 50-100, or all of the mRNA transcripts of the genes of Table A. In some embodiments, the at least one mRNA transcript is chosen from a group consisting of 2-120, 5-120, 10-120, 50-120, 80-120, 2-128, 5-128, 10-128, 50-128, 80-128, 5-50, 10-50, 50-100, or all of the mRNA transcripts of the genes of Table A. In some cases, the at least one mRNA transcript consists of 2-120, 5-120, 10-120, 50-120, 80-120, 2-128, 5-128, 10-128, 50-128, 80-128, 5-50, 10-50, 50-100, or all of the mRNA transcripts of the genes of Table A. Furthermore, in some embodiments, the at least one mRNA transcript comprises at least one mRNA that co-expresses with at least one gene listed in Table A, or that is found in the same biological or cell signaling pathway as a gene listed in Table A herein. As noted above, the term “mRNA transcript” as used herein indicates an mRNA obtained from a gene. Thus, each “mRNA transcript” herein is from a different gene, and a reference to two or more mRNA transcripts herein means the mRNA transcripts of two or more genes. Thus, if 2-150 mRNA transcripts are assayed herein, the mRNA transcripts are assayed to determine the expression at the RNA level of 2-150 different genes.

In some embodiments, the at least one mRNA transcript is chosen from a group consisting of 2-120, 5-120, 10-120, 50-120, 80-120, 2-128, 5-128, 10-128, 50-128, 80-128, 5-50, 10-50, 50-100, or all of the mRNA transcripts of the genes of Table A (i.e., mRNA transcripts of the genes listed in Table A) and at least one reference mRNA transcript. In some embodiments, the at least one mRNA transcript consists of 2-120, 5-120, 10-120, 50-120, 80-120, 2-128, 5-128, 10-128, 50-128, 80-128, 5-50, 10-50, 50-100, or all of the mRNA transcripts of the genes of Table A and at least one reference mRNA transcript or other reference RNA (such as a ribosomal RNA or other non-mRNA molecule). In such cases, the reference mRNA transcript or other reference RNA is not expected to significantly differ in expression between a sample from a patient with rejection and one without rejection. An example of such a reference mRNA transcript is the mRNA of a so-called housekeeping gene, for instance. Examples include, for instance, ACTB, B2M, UBC, GAPDH, HPRT1 and YWHAE. In some embodiments, a reference gene can comprise one or more of YWHAE, TTC5, C2orf44, or Chr3. In some embodiments, mRNA transcripts of a reference gene or genes are used to normalize the mRNA levels in the sample as a whole prior to analysis. In other embodiments, mRNA levels are normalized against the overall mRNA levels found in the sample. Normalization, for example, may help to control for the quality of the RNA of a sample, or the amount of the RNA of the recipient sample that is obtained.

In some embodiments, the at least one mRNA transcript whose expression level is assessed in the methods is chosen as an mRNA transcript whose expression significantly differs between solid organ transplant recipients with rejection compared to those without rejection. For example, the expression level of some mRNA transcripts may increase in the event of a rejection. In contrast, the expression level of some mRNA transcripts may decrease in the event of a rejection. In some cases, all of the assessed mRNA transcripts show an increase in expression level in the event of a rejection. In some cases, all of the assessed mRNA transcripts show a decrease in expression level in the event of a rejection. In yet other cases, some of the mRNA transcripts show an increase in expression levels in the event of a rejection, while others decrease in expression in the event of a rejection.

In some embodiments, the at least one mRNA transcript assessed in methods herein, and whose expression significantly differs between solid organ transplant recipients with rejection compared to those without rejection is of a gene involved in one or more of interferon gamma signaling. CD22-mediated BCR rejection, Rho GTPase signaling, or B cell receptor signaling. In some embodiments, such mRNA transcripts comprise transcripts of genes in one or more such pathways and also listed in Table A herein.

In some embodiments, an algorithm may be employed to determine an overall expression profile for the at least one mRNA transcript in the recipient and to compare that overall expression profile to those of exemplary expression profiles of the same mRNA transcripts in a reference sample of recipients with and without rejection. For example, in some embodiments, an algorithm may be developed that assesses such variables as the level of expression of from 2 to, for example 500, 1000, or 2000 different mRNA transcripts, and may group expression levels of mRNA transcripts of different types of genes from different biological pathways according to whether they increase or decrease with rejection, and the extent to which their levels change, and the overall importance of those pathways to the development of rejection. In some cases, a trained algorithm may be used, for example, that is adjusted and improved as more and more data from reference subjects is added to an underlying database from which the algorithm is developed. Particularly where several mRNA transcripts with different behaviors in development of rejection are used in the methods herein, an algorithm run by a computer system may be required to accurately determine whether a particular recipient's mRNA transcript expression profile indicates likelihood that the recipient has rejection or whether it indicates non-rejection. Thus, in some embodiments, a result of an algorithm is used to determine if a recipient has a gene expression profile indicating a likelihood of rejection.

In some embodiments, the expression level of the at least one mRNA transcript is determined by reverse transcription PCR (RT-PCR) (such as quantitative RT-PCR), hybridization to an array, or next generation sequencing. In some embodiments. mRNA transcript levels can be determined using a probe array. A number of distinct array formats are available. Some arrays, such as an Affymetrix HG-U133 PM microarray or other Affymetrix GeneChip R: array, have different probes occupying discrete known areas of a contiguous support. Exemplary microarrays include but are not limited to the Affymetrix Human Genome U133 Plus 2.0 GeneChip or the HT HG-U133+PM Array Plate. For example, the mRNA transcripts corresponding to the genes listed in Table A may be analyzed by hybridization based on the Probe Set ID provided in Table A, on the listed HT HG-U133+PM Array (Affymetrix) provided in the Table. Alternatively, if PCR is used, appropriate PCR probes may be used that hybridize to regions near the 5′ and 3′ ends of the mRNA transcripts for the genes, such as, for example 80-120 base pairs near each end of the transcript. In some cases, nested probes or combinations of more than 2 probes may also be used to detect mRNA transcripts for particular genes. Accordingly, the expression level of the at least one mRNA transcript herein may be determined in some embodiments from a complementary DNA (cDNA) obtained from the mRNA transcript, or a double stranded DNA amplicon obtained from the mRNA transcript.

An array contains one or more probes either perfectly complementary to a particular target mRNA transcript or sufficiently complementarity to the target mRNA transcript to distinguish it from other mRNA transcripts in the sample, and the presence of such a target mRNA transcript can be determined from the hybridization signal of such probes, optionally by comparison with mismatch or other control probes included in the array. In some cases, the target bears a fluorescent label, in which case hybridization intensity can be determined by, for example, a scanning confocal microscope in photon counting mode. Appropriate scanning devices are described by e.g., U.S. Pat. Nos. 5,578,832, and 5,631,734. The intensity of labeling of probes hybridizing to a particular mRNA transcript or its amplification product provides a raw measure of expression level.

In other methods. mRNA transcript levels can be determined by so-called “real time amplification” methods also known as quantitative PCR (qPCR or qRT-PCR) or Taqman. For example, an mRNA transcript is converted to the complementary DNA sequence (cDNA) by a reverse transcriptase, and the resulting cDNA is then amplified. The basis for this method of monitoring the formation of amplification product formed during a PCR reaction with a template using oligonucleotide probes/oligos specific for a region of the template to be detected. In some embodiments, qPCR or Taqman are used immediately following a reverse-transcriptase reaction performed on isolated cellular mRNA: this variety serves to quantitate the levels of individual mRNA transcripts during qPCR.

Taqman uses a dual-labeled fluorogenic oligonucleotide probe. The dual labeled fluorogenic probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye. Regardless of labelling or not, the qPCR probe is designed to have at least substantial sequence complementarity with a site on the target mRNA transcript or nucleic acid derived from. Upstream and downstream PCR primers that bind to flanking regions of the locus are also added to the reaction mixture. When the probe is intact, energy transfer between the two fluorophores occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector. The recorded values can then be used to calculate the increase in normalized reporter emission intensity on a continuous basis and ultimately quantify the amount of the mRNA transcript being amplified. mRNA transcript levels can also be measured without amplification by hybridization to a probe, for example, using a branched nucleic acid probe, such as a QuantiGene′R: Reagent System from Panomics.

Quantitative PCR (qPCR) can also be performed without a dual-labeled fluorogenic probe by using a fluorescent dye (e.g. SYBR Green) specific for dsDNA that reflects the accumulation of dsDNA amplified specific upstream and downstream oligonucleotide primers. The increase in fluorescence during the amplification reaction is followed on a continuous basis and can be used to quantify the amount of mRNA transcript being amplified. qPCR can also be performed using microfluidics technology or digital-droplet PCR.

For qPCR or Taqman, the levels of particular genes may be expressed relative to one or more reference genes measured from the same sample using the same detection methodology. Examples include, for instance, ACTB, B2M, UBC, GAPDH, HPRT1 and YWHAE. In some embodiments, a reference gene can comprise one or more of YWHAE, TTC5, C2orf44, or Chr3.

In some embodiments, for qPCR or Taqman detection, a “pre-amplification” step is performed on cDNA transcribed from cellular RNA prior to the quantitatively monitored PCR reaction. This serves to increase signal in conditions where the natural level of the RNA/cDNA to be detected is very low. Suitable methods for pre-amplification include but are not limited LM-PCR, PCR with random oligonucleotide primers (e.g. random hexamer PCR), PCR with poly-A specific primers, and any combination thereof.

In other methods, gene or nucleic acid levels can be determined by sequencing, such as by DNA sequencing. Sequencing may be performed by any available method or technique. Sequencing methods may include: Next Generation sequencing, high-throughput sequencing, pyrosequencing, classic Sanger sequencing methods, sequencing-by-ligation, sequencing by synthesis, sequencing-by-hybridization, RNA-Seq (Illumina), Digital Gene Expression (Helicos), next generation sequencing, single molecule sequencing by synthesis (SMSS) (Helicos), Ion Torrent Sequencing Machine (Life Technologies/Thermo-Fisher), massively-parallel sequencing, clonal single molecule Array (Solexa), shotgun sequencing, single molecule nanopore sequencing, sequencing by ligation, sequencing by hybridization, sequencing by nanopore current restriction, Maxim-Gilbert sequencing, primer walking, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing. pH-mediated sequencing, semiconductor sequencing or a combination thereof. Conducting one or more sequencing reactions may comprise whole genome sequencing or exome sequencing.

Sequencing reactions may comprise one or more capture probes or libraries of capture probes. At least one of the one or more capture probe libraries may comprise one or more capture probes to 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250 or more genomic regions. The libraries of capture probes may be at least partially complementary. The libraries of capture probes may be fully complementary. The libraries of capture probes may be at least about 5%, 10%, 15%, 20%, %, 25%, 30%, 35%, 40%, 45%, 50%, 55%, 60%, 70%, 80%, 90%, 95%, 97% or more complementary.

In some embodiments, where the recipient is a kidney transplant recipient, methods used to determine level of the at least one mRNA transcript are derived from those described in U.S. Pat. No. 10,443,100 B2, which is incorporated herein by reference. In some embodiments, where the recipient is a kidney transplant recipient, a commercial assay and algorithm such as a TruGraf® assay (Eurofins—Transplant Genomics, Framingham, MA) may be used to determine the level of the at least one mRNA transcript and whether the recipient's gene expression profile indicates likelihood of rejection on the basis of an algorithm result. In some embodiments, a result of a TruGraf® Gene Expression Profile (GEP) probability score algorithm is used. This algorithm provides results scaled on a 0-1 scale with scores >0.50 being considered positive (i.e., indicating rejection) and scores <0.50 being negative, i.e., indicating no rejection).

### Dd-cfDNA Determination Methods

Methods herein also involve determining dd-cfDNA in the sample, and, in particular, whether or not the level of dd-cfDNA, such as the percent dd-cfDNA out of total cfDNA in the sample, is at or above a particular pre-determined threshold indicating rejection.

There are several different methods for determining dd-cfDNA in a sample. In some methods, dd-cfDNA is determined by using genotyping data from both the donor and the recipient, for example, each obtained prior to the transplantation. In many other cases however, donor genotype data is not available. Thus, in some cases, only recipient genotype data is available and used in the method. For example, recipient genotyping may be performed on PBMC samples from the recipient. In yet other cases, neither the donor nor the recipient has been genotyped prior to determining dd-cfDNA. In some embodiments, the pre-determined threshold of dd-cfDNA is ≥ 0.5%, ≥ 0.6%, ≥ 0.7%, ≥ 0.8%, ≥ 0.9%, ≥ 1%, ≥ 1.2%, ≥ 1.5%, or ≥ 2%.

The pre-determined threshold at or above which a recipient is indicated to have a rejection may vary depending upon the amount of genotype data that is available and used in the determination. For example, donor and/or recipient genotype data is not always available for use in algorithms developed to determine the percent dd-cfDNA. Where both are available, a pre-determined threshold may be relatively low, such as ≥ 0.5%, ≥ 0.6%, ≥ 0.7%, ≥ 0.8%, ≥ 0.9%, ≥ 1% indicating rejection, as fewer assumptions are required in the method of determination. In addition, where recipient genotype data is available, in some embodiments a pre-determined threshold of ≥ 0.5%, ≥0.6%, ≥0.7%, ≥0.8%, ≥0.9%, ≥1% indicates rejection. In particular cases, a pre-determined threshold of ≥ 0.7% indicates rejection, such as when recipient genotype data are available. Accordingly, an “amount of dd-cfDNA” or “level of dd-cfDNA” may in some embodiments be reported as a percentage of the total cfDNA obtained from the sample.

In some embodiments, dd-cfDNA is determined by analysis of SNPs in the cfDNA obtained from the sample. For example, a donor and a recipient may have certain different SNPs at particular genetic loci. Where donor and/or recipient genotype data are available. i.e., a “two-genome” approach, particular SNP differences may be known prior to analysis. Alternatively, where genotype data for the donor and/or the recipient are not available, particular SNP differences may be found based on assaying for unique SNPs that occur in subjects with the same disease as the recipient, such as kidney disease, lung disease, liver disease, pancreatitis or pancreatic disease, or heart disease, with the expectation that the donor cfDNA will not show these unique SNPs. In cases where recipient and donor genotype data are not available, a higher threshold may be pre-determined for a recipient to show rejection, such as, for example, ≥ 1%, ≥ 1.2%, ≥ 1.5%, or ≥ 2%. In some embodiments, a particular threshold is pre-determined based on clinical studies that compare predictions of rejection based on the specific dd-cfDNA analysis algorithm used to determine the percent dd-cfDNA to actual rejection based on a surveillance biopsy result.

In a “two genomes” method that includes both recipient and donor genotype information, it may only be necessary to assay SNPs that are homozygous but differ between recipient and donor. In an approach that does not rely on donor genotype information, to quantify the observed abundance of alleles of each genotyped SNP in cfDNA sequences by sequencing, low quality reads, reads that are not mapped uniquely to the genome, and reads with potential for mapping biased by genetic variability may be filtered. Duplicated reads are then removed and allele appearances of each genotyped SNP counted (e.g. by a SAMtools mpileup function). The observed allele appearances in cfDNA and the recipient genotype are the inputs for a “one-genome” model.

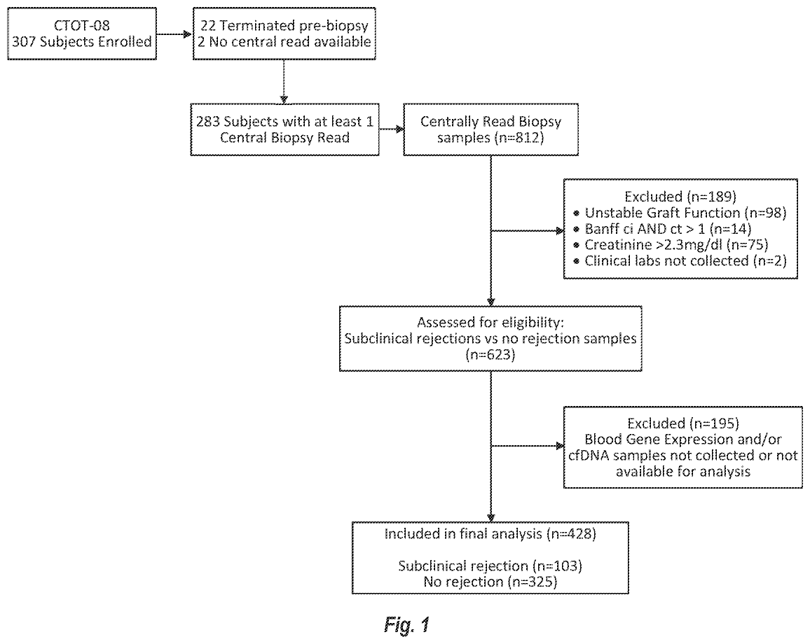

In such a one-genome model, to calculate the probability of the observed cfDNA, the probability of each possible donor and recipient genotype are first calculated. Recipient genotype can depend on the recipient measured genotype and the genotyping error rate. Since vital organ transplants are rarely closely related, the model can assume that the donor genotype is randomly selected from a human population. Given this assumption, the probability of a specific donor allele is its frequency in the population. The algorithm, in some embodiments, then iterates over the 1000 Genomes Project populations and super-populations (available from the International Genome Sample Resource (IGSR)) to detect the most likely ancestral population of the donor. To clarify further, the probability of observing a specific allele in a cfDNA fragment is computed by integrating over all possible recipient and donor genotypes and depends on the sequencing error rate, the fraction of dd-cfDNA in the recipient plasma and the probabilities of observing the allele conditioning on it being donor- or recipient-derived (FIG. 1A indicated (a)). Finally, the log-likelihood of the data is computed by summing log-likelihoods over all SNPs, assuming SNPs are independent (this assumption is also made by the two-genomes method). An optimization algorithm is then used to find the maximum likelihood parameter values.

In some instances, this procedure can be executed in a parallelized fashion, dramatically speeding up the determination of dd-cfNA in multiple samples or sequencing reactions (e.g. from the same individual or from multiple individuals).

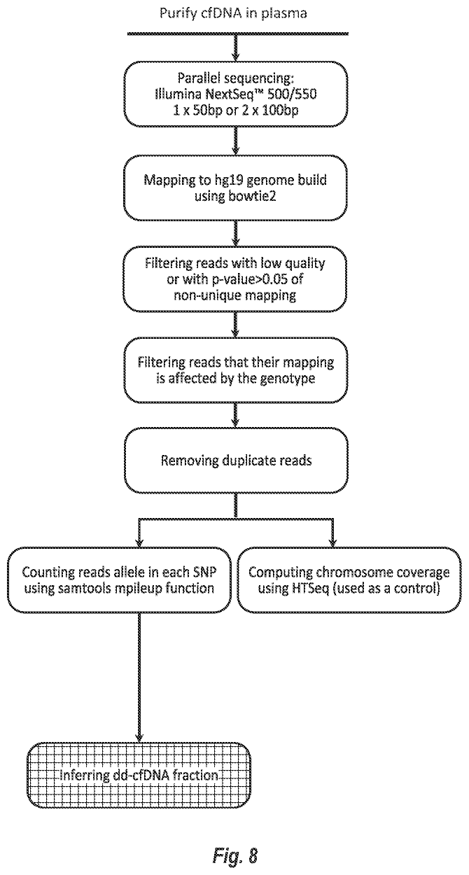

An example of a “one-genome” dd-cfDNA determination without donor genotype information is as shown in FIG. 8, and an associated algorithm for determining the % dd-cfDNA is described, for example, in US Patent Application Publication No. US2021/0115506 A1 and WO2018187226A1, which are each incorporated in their entirety by reference herein. Certain commercial dd-cfDNA assays, such as a Viracor TRAC® assay (Eurofins—Viracor, Lenexa, KS, USA), AlloSure® assay (CareDx), Prospera® assay (Natera), or TheraSure® assay (Oncocyte), may also be used in some embodiments.

In some embodiments herein, methods comprise determining the level of donor derived cell-free DNA (dd-cfDNA) in the cfDNA and the expression level of the at least one mRNA transcript in the recipient's sample, and distinguishing rejection from non-rejection in the recipient based upon results from an algorithm that considers both the dd-cfDNA and the expression level of at least one mRNA transcript and that provides a result indicating rejection or non-rejection. Thus, for example, a trained algorithm may be used that accounts for both the dd-cfDNA level and the mRNA transcript expression data collectively (i.e., in one algorithm generating one score result) rather than separately.

### Trained Algorithms

In some embodiments, methods include using a trained algorithm to analyze sample data, particularly to detect or rule-out rejection. In some embodiments, methods comprise applying a trained algorithm to the expression level of the at least one mRNA transcript and determining a result of the algorithm, wherein the result indicates rejection or non-rejection. In some embodiments, the level of dd-cfDNA is determined using a trained algorithm. A “trained algorithm” or “training algorithm,” as used herein, is an algorithm that is developed based on a set of training data, such as mRNA transcript expression levels of particular genes in subjects with or without rejection, such as tens or hundreds of such genes, or such as SNP information for SNPs throughout a genome that may differ between a donor and recipient, and developed to use the data to distinguish data profiles associated with different outcomes or phenotypes, such as rejection and non-rejection.

In such supervised learning approaches, a group of samples from two or more groups (e.g. rejection and non-rejection, as well as types of rejection such as acute cellular rejection and antibody mediated rejection) are analyzed with a statistical classification method. Differential gene or nucleic acid level data can be discovered that can be used to build a classifier that differentiates between the two or more groups, such as rejection and non-rejection. A new sample can then be analyzed so that the classifier can associate the new sample with one of the two or more groups. Examples of trained algorithms include without limitation a neural network (multi-layer perceptron), support vector machine, k-nearest neighbors, Gaussian mixture model, Gaussian, naive Bayes, decision tree and radial basis function (RBF). Linear classification methods include Fisher's linear discriminant, LDA, logistic regression, naive Bayes classifier, perceptron, and support vector machines (SVMs). Other algorithm methods compatible with the invention include quadratic classifiers, k-nearest neighbor, boosting, decision trees, random forests, neural networks, pattern recognition, Elastic Net, Golub Classifier, Parzen-window, Iterative RELIEF, Classification Tree, Maximum Likelihood Classifier, Nearest Centroid, Prediction Analysis of Microarrays (PAM), Fuzzy C-Means Clustering, Bayesian networks and Hidden Markov models.

Classification by a trained algorithm using supervised methods is performed in some embodiments by the following methodology:

In order to solve a given problem of supervised learning, one can consider various steps:

1. Gather a training set. These can include, for example, samples that are from recipients with known rejection and with known non-rejection, and in some cases also normal subjects, and/or subjects with particular types of rejection such as acute cellular rejection and antibody mediated rejection. These training samples are used to “train” the classifier.

2. Determine the input “feature” representation of the learned function. The accuracy of the learned function depends on how the input object is represented. Typically, the input object is transformed into a feature vector, which contains a number of features that are descriptive of the object. The number of features should not be too large, because of the curse of dimensionality: but should be large enough to accurately predict the output.

3. Determine the structure of the learned function and corresponding learning algorithm. A learning algorithm is chosen, e.g., artificial neural networks, decision trees, Bayes classifiers or support vector machines. The learning algorithm is used to build the classifier.

4. Build the classifier (e.g. classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set.

Once the classifier (e.g. classification model) is determined as described above, it can be used to classify a sample, e.g., that of a solid organ transplant recipient analyzed by the methods of the invention, or expression levels of particular mRNA transcripts from such a recipient.

Training of multi-dimensional algorithms may be performed using numerous samples. For example, training may be performed using at least about 10, 20, 30, 40, 50, 60, 70, 80, 90, 100, 110, 120, 130, 140, 150, 160, 170, 180, 190, 200 or more samples from subjects with known rejection or non-rejection outcomes. In some cases, training of the multi-dimensional algorithms may be performed using at least about 200, 210, 220, 230, 240, 250, 260, 270, 280, 290, 300, 350, 400, 450, 500 or more samples. In some cases, training may be performed using at least about 525, 550, 600, 650, 700, 750, 800, 850, 900, 950, 1000, 1100, 1200, 1300, 1400, 1500, 1600, 1700, 1800, 2000 or more samples.

For example, in some embodiments a trained algorithm for analyzing mRNA transcript expression data for, for example, tens or hundreds of different mRNA transcripts can be developed from a training data set of gene expression information from, for instance, several hundred transplant recipient subject samples with known rejection or non-rejection phenotypes. A Random Forest model may be trained on the dataset of the mRNA transcript levels from each subject of the dataset to generate a phenotypic classification/interpretation that predicts rejection or non-rejection in the training samples. That model may then be applied to a new sample of mRNA transcript data from a recipient whose rejection or non-rejection is unknown, providing a result indicating rejection or non-rejection for that recipient.

As trained algorithms require manipulation of many parameters simultaneously, often tens or hundreds of parameters, tracking SNPs or mRNA transcript levels, for example, they are developed and calculated using appropriate software programming methods, and may be implemented on a computer. A further discussion of computer and software implements that may be used to compute or develop a trained algorithm is provided further below.

### Distinguishing Likelihood of Different Types of Subclinical Rejection

As described in the Examples below, the present inventors discovered that the dd-cfDNA and mRNA transcript expression based assays for assessing likelihood of rejection are not redundant and, in fact, tend to correlate with different types of subclinical rejection. Specifically, the gene expression profile from analysis of mRNA transcripts preferentially detects acute cellular rejection while the dd-cfDNA assay preferentially detects antibody mediated rejection. Thus, for example, recipients with a positive result, indicating rejection, in one but not both of the assays may be more likely to have the type of rejection associated with that assay (e.g., acute cellular rejection or antibody-mediated rejection). Furthermore, use of the combined assay methods disclosed herein may assist in identifying subjects with early acute cellular rejection, which may precede a later antibody mediated rejection, allowing for therapeutic intervention that might help to reduce or inhibit an antibody mediated rejection. Accordingly, in some embodiments, the methods herein are capable of further distinguishing likelihood of acute cellular rejection from antibody-mediated rejection, wherein the dd-cfDNA level indicates presence or absence of antibody-mediated rejection, and wherein the level of the at least one mRNA transcript indicates presence or absence of acute cellular rejection.

### Methods of Treating Transplant Recipients

In some instances, the methods described herein provide information to a medical practitioner that can be useful in making a therapeutic decision. Therapeutic decisions may include decisions to: continue with a particular therapy, modify a particular therapy, alter the dosage of a particular therapy, stop or terminate a particular therapy, altering the frequency of a therapy, introduce a new therapy, introduce a new therapy to be used in combination with a current therapy, or any combination of the above. Furthermore, the methods used in this disclosure may guide the decision points in treatment regimens (e.g. addition of agents to the immunosuppression regimen due to increased evaluation of risk). For example, they may allow the evaluation of a patient with low time-of-transplant risk factors (e.g. high HLA matching between recipient and donor organ), or as having rejection, or non-rejection, justifying the adjustment of a treatment regimen.

In particular embodiments, methods herein may be used to determine whether a recipient of a solid organ transplant should or should not receive a surveillance biopsy. For example, in some embodiments, a recipient with a non-rejection result according to the methods herein, such as a negative result in both the dd-cfDNA and mRNA transcript expression analyses, may be determined to not be in need of a biopsy, whereas a recipient who is positive in one or both analyses may be determined to need a biopsy.

Thus, methods herein also include methods of treating a solid organ transplant recipient, wherein the recipient is determined to have a likelihood of rejection according to a method of distinguishing rejection from non-rejection herein. In some embodiments, the recipient is receiving at least one immunosuppressive drug. In some embodiments, the treatment method comprises increasing the frequency or dosage of the at least one immunosuppressant drug, administering a further immunosuppressant drug, or administering a different immunosuppressive drug to the recipient if the recipient has a positive result (indicating rejection) in the method of distinguishing rejection from non-rejection. In some cases, if the recipient's test results indicate rejection on either or both of the dd-cfDNA and mRNA transcript expression analyses, the method of treatment comprises performing a surveillance biopsy. In some cases, the dd-cfDNA and mRNA transcript expression analyses are performed prior to a surveillance biopsy and such a biopsy is not ordered for the recipient unless one or both tests provide a positive result. In some cases, the recipient does not show clinical signs of rejection at the time that the recipient's sample is obtained for the dd-cfDNA and mRNA transcript expression analyses to be performed.

Many different drugs are available for treating solid organ transplant rejection, such as immunosuppressive drugs used to treat transplant rejection, such as calcineurin inhibitors (e.g., cyclosporine, tacrolimus), mTOR inhibitors (e.g., sirolimus and everolimus), anti-proliferatives (e.g., azathioprine, mycophenolic acid, mycophenolate mofetil or MMF), corticosteroids (e.g., prednisone, prednisolone, and hydrocortisone), antibodies (e.g., rituximab, basiliximab, daclizumab, muromonab-CD3, alemtuzumab, anti-thymocyte globulin and anti-lymphocyte globulin), intravenous or subcutaneous immunoglobulins (IVIG), rabbit antithymocyte globulin (rATG), interleukin 2 (IL2) receptor antagonists (e.g. basiliximab or daclizumab), and biologics (e.g. belatacept), or combinations of one or more of the above. In some embodiments, a recipient may be receiving a standard of care treatment post-transplant.

An additional immunosuppressant regimen to note is a “breakout” regimen used for treatment of any rejection episodes that occur after organ transplant. This may be a permanent adjustment to the maintenance regimen or temporary drug therapy used to minimize damage during the acute rejection episode. The adjustment may comprise temporary or long-term addition of a corticosteroid, temporary use of lymphocyte-depleting agents, and long-term addition of antiproliferative agents (e.g. mycophenolate mofetil/MMF or azathioprine, for patients not already receiving it), and any combination thereof. Treatment may also comprise plasma exchange, intravenous immunoglobulin, and anti-CD-20 antibody therapy, and any combination thereof.

With respect to immunosuppression therapy of kidney transplant recipients, the 2009 Kidney Disease: Improving Global Outcomes (KDIGO) guidelines (see e.g. Kasiske et al. Am J Transplant. 2009 November; 9 Suppl 3:S1-155, which is incorporated by reference herein) outline an example immunosuppression regimen for a kidney transplant recipient. Prior to transplant, a patient receives an “induction” combination of immunosuppressants, ideally comprising a biologic agent such as an IL-2 receptor antagonist (e.g. basiliximab or daclizumab) or a lymphocyte-depleting agent (e.g. antithymocyte globulin, antilymphocyte globulin, alemtuzumab, and/or monomurab-CD3), which may be continued immediately after transplantation. The use of a lymphocyte-depleting agent may be recommended for patients considered at high risk of immune-mediated rejection. Calcineurin inhibitors (CNIs. e.g. tacrolimus) may be additionally used in the “induction” phase. After transplant, a patient may be treated with an initial maintenance immunosuppression regimen which ideally comprises a calcineurin inhibitor (e.g. tacrolimus) or an mTOR inhibitor (e.g. sirolimus) and an antiproliferative agent (e.g. mycophenolate mofetil or MMF). The initial maintenance regimen may optionally additionally comprise a corticosteroid. Within 2-4 months after transplantation with no acute rejection, the immunosuppression regimen may be adjusted to a long-term maintenance phase, where the lowest planned doses of immunosuppressants are used, calcineurin inhibitor therapy is continued (if originally used), and corticosteroid therapy is continued (if used beyond the first week of transplant).

In some embodiments, a method of treatment herein, if the recipient is negative in both of the dd-cfDNA and mRNA transcript expression analyses, indicating no rejection, comprises monitoring the recipient, including re-performing the tests at regular intervals, such as 1 week, 2 weeks, 3 weeks, 4 weeks, 2 months, 3 months, 4 months, 5 months, or 6 months, as part of a plan of active surveillance. “Active surveillance” herein refers to a treatment plan comprising regular physician visits, and optionally, regular diagnostic testing, to monitor a recipient for signs of rejection and/or organ dysfunction over a period of time. In some cases, the subject may be receiving immunosuppressive therapy, while in other cases the recipient may not be receiving therapeutics. For example, if rejection is not detected according to the dd-cfDNA and mRNA transcript expression methods herein, suitable active surveillance methods of treatment may include refraining from biopsy procedures or immunosuppressant regimen adjustments for a specific period of time, such as e.g. 1 week, 2 weeks, 3 weeks, 4 weeks, 2 months, 3 months, 4 months, 5 months, or 6 months. In some cases, where a recipient is receiving immunosuppressive therapy and the methods herein indicate non-rejection, the current immunosuppressive therapy may be maintained, or may be reduced, such as through administration of a lower dose of the current drugs or by an alteration in the drugs being administered. In some cases, when rejection is not detected and the patient has previously received an increase in dose of a particular immunosuppressant of their regimen, the current increase in dose or new immunosuppressant administration may be maintained or reduced.

In some methods, expression levels are determined at intervals in a particular patient (i.e., monitoring). Preferably, the monitoring is conducted by serial minimally-invasive tests such as blood draws: but, in some cases, the monitoring may also involve analyzing a kidney biopsy, either histologically or by analyzing a molecular profile. The monitoring may occur at different intervals, for example the monitoring may be hourly, daily, weekly, monthly, yearly, or some other time period, such as twice a month, three times a month, every two months, every three months, every 4 months, every 5 months, every 6 months, every 7 months, every 8 months, every 9 months, every 10 months, every 11 months, or every 12 months.

For example, if methods herein are conducted on a regular basis, they can provide an indication whether an existing immunosuppressive regimen is working, whether the immunosuppressive regimen should be changed (e.g. via administration of a new immunosuppressant to the transplant recipient or increase in dose of an immunosuppressant currently being administered to the transplant recipient) or whether a biopsy or increased monitoring by other rejection markers such as creatinine or glomerular filtration rate should be performed. In some cases, consecutive (e.g. at least two) tests positive for rejection as described herein indicate that an additional action be taken, e.g. adjustment of the immunosuppressive regimen (e.g. via administration of a new immunosuppressant to the transplant recipient or increase in dose of an immunosuppressant currently being administered to the transplant recipient), collection and evaluation of a biopsy, further biomarker testing such as (in a kidney transplant recipient) administration of a serum creatinine and/or eGFR test. In some cases, consecutive (e.g. at least two, three, four, five, six, seven, eight, nine, ten) tests ambiguous for rejection vs. non-rejection as described herein may indicate that an additional confirmatory action be taken. e.g. collection and evaluation of a biopsy or further biomarker testing such as (in a kidney transplant recipient) administration of a serum creatinine and/or eGFR test. The consecutive (e.g. at least two, three, four, five, six, seven, eight, nine, ten) tests may be separated by an appropriate time period (e.g. one day, one week, two weeks, three weeks, one month, two months, three months, four months, five months, six months, or one year) to ensure that the tests accurately represent a trend.

Treatment methods provided herein include administering a blood test (e.g., a test to detect subclinical acute rejection) to a transplant recipient who has already undergone a surveillance biopsy of the kidney and received a biopsy result in the form of a histological analysis or a molecular profiling analysis. In some particular instances, the analysis of the biopsy (e.g., by histology or molecular profiling) may result in ambiguous, inconclusive or borderline results. In such cases, a blood test provided herein may assist a caregiver with determining whether the transplant recipient has subclinical acute rejection or with interpreting the biopsy. In other cases, the biopsy itself may be inconclusive or ambiguous, and in such cases the molecular analysis of the biopsy may be used in adjunct with the histology to confirm a diagnosis. In some instances, the analysis of the biopsy may yield a negative result. In such cases, the subject may receive a dd-cfDNA and mRNA transcript expression analysis as provided herein in order to confirm the negative result, or to detect subclinical acute rejection. In some cases, after receiving any type of biopsy result (e.g., negative result, ambiguous, inconclusive, borderline, positive), the patient may receive multiple, serial dd-cfDNA and mRNA transcript expression analyses as described herein, in order to monitor changes in molecular markers correlated with subclinical acute rejection.

Treatment methods provided herein also include performing a biopsy on a transplant recipient who has received a dd-cfDNA and mRNA transcript expression analysis as described herein. In some embodiments, the recipient is positive for both the dd-cfDNA and mRNA transcript expression analysis portions of the methods, indicating rejection. In other cases, the recipient is positive only for dd-cfDNA or for mRNA transcript expression results. In cases where only the dd-cfDNA or mRNA transcript expression yields a positive result, or where such result is borderline (i.e., at or near the threshold separating rejection from non-rejection), the patient's healthcare worker may use the results of a biopsy test as a complement in order to confirm whether rejection is present.

### Computer Implemented Methods and Systems for Conducting Methods Herein

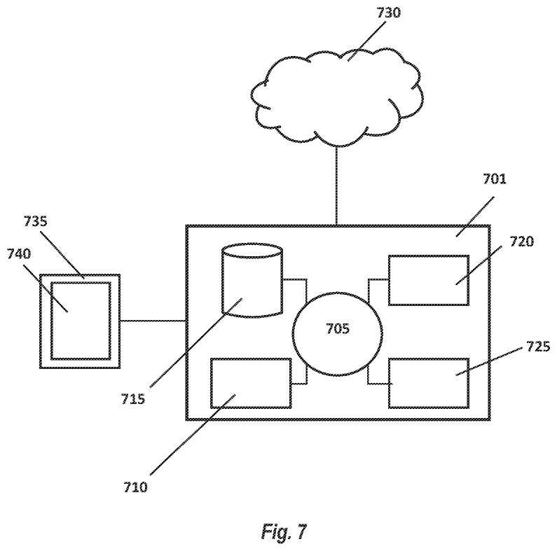

As described previously, gene or nucleic acid levels can be analyzed and associated with status of a subject (e.g., presence or absence of rejection) in a digital computer, while algorithms herein, such as trained algorithms may be applied through use of a computer. As shown in FIG. 7, in some embodiments, a sample (710) is first collected from a subject (for example, from a transplant recipient). The sample is assayed (720) and nucleic acid products are generated. A computer system (730) is used in analyzing the data and making a classification of rejection or non-rejection (740) based on, for example, results from both the dd-cfDNA and the expression level of at least one mRNA transcript, wherein rejection in the recipient is indicated by either or both of (i) a level of dd-cfDNA at or above a pre-determined threshold value, and (ii) expression level of the at least one mRNA transcript or a result of an algorithm based on the expression level indicating rejection, or alternatively, wherein rejection in the recipient is indicated by result of an algorithm accounting for both the dd-cfDNA level and the mRNA transcript expression level data.

In some embodiments of the disclosure, a system that is capable of determining the level of each of dd-cfDNA and expression of the at least one mRNA transcript is used to conduct methods herein. In some cases, such a system may include components for conducting assays to determine the level of one or both of dd-cfDNA level and expression level of the at least one mRNA transcript. In some cases, alternatively or additionally, a system may include a computer and appropriate software for conducting one or more algorithms, such as trained algorithms, in order to determine the level of dd-cfDNA and expression of at least one mRNA transcript from a recipient sample. A system may comprise software that provides an algorithm result for a recipient, for example, positive or negative (i.e. rejection or no rejection), for each of the dd-cfDNA and mRNA transcript expression analyses, or for both analyses in combination, which may then in some embodiments be provided to a caregiver for the recipient in order to determine further treatment steps for the recipient.

Optionally, in a system herein, a computer is directly linked to a scanner or the like receiving experimentally determined signals related to gene or nucleic acid levels, (i.e., for SNP identification or identification of the levels of various expressed mRNA transcripts), and the like. Alternatively, gene or nucleic acid levels can be input by other means. The computer can be programmed to convert raw signals into gene or nucleic acid levels (absolute or relative), compare measured gene or nucleic acid levels with one or more reference levels, or a scale of such values, as described above. The computer can also be programmed to assign values or other designations to gene or nucleic acid levels based on the comparison with one or more reference gene or nucleic acid levels, and to aggregate such values or designations for multiple gene or nucleic acids in a profile. The computer can also be programmed to output a value or other designation providing an indication of rejection or non-rejection as well as any of the raw or intermediate data used in determining such a value or designation.

The methods, systems, kits and compositions provided herein may also be capable of generating and transmitting results through a computer network. As shown in FIG. 7, a sample 720 is first collected from a subject (e.g. transplant recipient, 710). The sample is assayed 730 and gene or nucleic acid levels are generated. A computer system 740 is used in analyzing the data and making classification of the sample. The result is capable of being transmitted to different types of end users via a computer network. In some instances, the subject (e.g. patient) may be able to access the result by using a standalone software and/or a web-based application on a local computer capable of accessing the internet. In some instances, the result can be accessed via a mobile application provided to a mobile digital processing device (e.g. mobile phone, tablet, etc.). In some instances, the result may be accessed by physicians and help them identify and track conditions of their patients. In some instances, the result may be used for other purposes such as education and research.

The methods, kits, and systems disclosed herein may include at least one computer program, or use of the same. A computer program may include a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. The computer program will normally provide a sequence of instructions from one location or a plurality of locations. In various embodiments, a computer program includes, in part or in whole, one or more web applications, one or more mobile applications, one or more standalone applications, one or more web browser plug-ins, extensions, add-ins, or add-ons, or combinations thereof.

Further disclosed herein are systems for classifying one or more samples and uses thereof. The system may comprise (a) a digital processing device comprising an operating system configured to perform executable instructions and a memory device: (b) a computer program including instructions executable by the digital processing device to classify a sample from a subject comprising: (i) a first software module configured to receive a an gene or nucleic acid level profile of one or more genes from the sample from the subject; (ii) a second software module configured to analyze the gene or nucleic acid level profile from the subject: and (iii) a third software module configured to classify the sample from the subject based on a classification system comprising two or more classes (e.g. rejection vs. non-rejection).

FIG. 7 shows a computer system (also “system” herein) 701 programmed or otherwise configured for implementing the methods of the disclosure, such as producing a selector set and/or for data analysis. The system 701 includes a central processing unit (CPU, also “processor” and “computer processor” herein) 705, which can be a single core or multi core processor, or a plurality of processors for parallel processing The system 701 also includes memory 710 (e.g., random-access memory, read-only memory, flash memory), electronic storage unit 715 (e.g., hard disk), communications interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communications bus (solid lines), such as a motherboard. The storage unit 715 can be a data storage unit (or data repository) for storing data. The system 701 is operatively coupled to a computer network (“network”) 730 with the aid of the communications interface 720. The network 730 can be the Internet, an internet and/or extranet, or an intranet and/or extranet that is in communication with the Internet. The network 730 in some instances is a telecommunication and/or data network. The network 730 can include one or more computer servers, which can enable distributed computing, such as cloud computing. The network 730 in some instances, with the aid of the system 701, can implement a peer-to-peer network, which may enable devices coupled to the system 701 to behave as a client or a server.

The system 701 is in communication with a processing system 735. The processing system 735 can be configured to implement the methods disclosed herein. In some examples, the processing system 735 is a microarray scanner. In some examples, the processing system 535 is a real-time PCR machine (optionally microfluidic). In some examples, the processing system 735 is a nucleic acid sequencing system, such as, for example, a next generation sequencing system (e.g., Illumina sequencer, Ion Torrent sequencer, Pacific Biosciences sequencer. BGI sequencing system). The processing system 735 can be in communication with the system 701 through the network 730, or by direct (e.g., wired, wireless) connection. The processing system 735 can be configured for analysis, such as nucleic acid sequence analysis.

Methods as described herein can be implemented by way of machine (or computer processor) executable code (or software) stored on an electronic storage location of the system 701, such as, for example, on the memory 710 or electronic storage unit 715. During use, the code can be executed by the processor 705. In some examples, the code can be retrieved from the storage unit 715 and stored on the memory 710 for ready access by the processor 705. In some situations, the electronic storage unit 715 can be precluded, and machine-executable instructions are stored on memory 710.

### Digital Processing Device

Systems herein for conducting the methods may include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles.

The digital processing device will normally include an operating system configured to perform executable instructions. The operating system is, for example, software, including programs and data, which manages the device's hardware and provides services for execution of applications. Exemplary operating systems include, by way of non-limiting examples, FreeBSD, OpenBSD, NetBSD®, Linux, Apple® Mac OS X Server®, Oracle® Solaris®, Windows Server®, and Novell® NetWare®, as well as the personal computer operating systems such as Microsoft® Windows®, Apple® Mac OS X®, UNIX®, and UNIX-like operating systems such as GNU/Linux®. In some embodiments, the operating system is provided by cloud computing. Mobile smart phone operating systems include, by way of non-limiting examples, Nokia® Symbian® OS, Apple® iOS®, Research In Motion® BlackBerry OS®, Google Android®, Microsoft® Windows Phone® OS, Microsoft® Windows Mobile® OS, Linux®, and Palm® WebOS®.

The digital processing device generally includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, or other external memory devices.

A digital processing device may also include a display to send visual information to a user. The digital processing device may include an input device to receive information from a user, e.g., from a keyboard or touch screen or other means of inputting information.

### Computer Programs

The methods, kits, and systems disclosed herein may include one or more non-transitory computer readable storage media encoded with a program including instructions executable by the operating system to perform and analyze the test described herein; preferably connected to a networked digital processing device. A non-transitory computer-readable storage media may be encoded with a computer program including instructions executable by a processor to create or use an algorithm to determine one or more results for methods herein (i.e. levels of dd-cfDNA or parameters needed to determine level of dd-cfDNA, and/or levels of particular mRNA transcripts, or expression profiles from the at least one mRNA transcript). The storage media may comprise a database, in a computer memory, of one or more clinical features of control samples, for example, or of other data or parameters used in algorithms of the methods, or in creating a trained algorithm. In some embodiments, a computer program includes a web application. In light of the disclosure provided herein, those of skill in the art will recognize that a web application, in various embodiments, utilizes one or more software frameworks such as Microsoft® .NET or Ruby on Rails (RoR), and one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

In some embodiments, the computer program includes a web browser plug-in. In computing, a plug-in is one or more software components that add specific functionality to a larger software application. Examples of web browser plug-ins include Adobe® Flash® Player, Microsoft® Silverlight®, and Apple® QuickTime®.

Software modules connected with method herein may be created by techniques known to those of skill in the art using machines, software, and languages known to the art.

### Databases

The methods, kits, and systems disclosed herein may comprise one or more databases, or use of the same. Exemplary databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Data Transmission and Reports

Methods herein may further comprise providing one or more reports, and systems herein for conducting such methods may include means for generating such reports. The one or more reports may comprise a status or outcome of a transplant in a subject, i.e. whether the method indicates rejection or non-rejection. The one or more reports may also comprise information pertaining to therapeutic regimens for use in treating transplant rejection or in suppressing an immune response in a recipient, such as based on the results of the method. The one or more reports may be transmitted to a recipient or to a medical representative of the recipient such as a physician, physician's assistant, nurse, or other medical personnel, or to a family member, guardian, or legal representative of the subject.

### Exemplary Embodiments

Exemplary embodiments herein include the following:

1. A method of distinguishing rejection from non-rejection in a solid organ transplant recipient, the method comprising
a. obtaining a sample from the solid organ transplant recipient;
b. obtaining cell-free DNA (cfDNA) and mRNA from the sample;
c. determining (i) the level of donor derived cell-free DNA (dd-cfDNA) in the cfDNA and (ii) the expression level of at least one mRNA transcript, wherein the at least one mRNA transcript shows significantly different expression levels in transplant rejection compared to transplant non-rejection subjects: and
d. distinguishing rejection from non-rejection in the recipient based upon results from both the level of the dd-cfDNA and the expression level of at least one mRNA transcript, wherein rejection in the recipient is indicated by either or both of (i) a level of dd-cfDNA at or above a pre-determined threshold value, and (ii) expression level of the at least one mRNA transcript or a result of an algorithm based on the expression level indicating rejection.
   2. The method of embodiment 1, wherein distinguishing rejection from non-rejection comprises applying a trained algorithm to the expression level of the at least one mRNA transcript and determining a result of the algorithm, wherein the result indicates rejection or non-rejection.
   3. The method of embodiment 1 or 2, wherein rejection in the recipient is indicated by a pre-determined threshold value of dd-cfDNA of ≥ 0.5%, ≥ 0.6%, ≥ 0.7%, ≥ 0.8%, ≥ 0.9%, ≥ 1%, ≥ 1.2%, ≥ 1.5%, or ≥ 2%.
   4. The method of embodiment 3, wherein rejection in the recipient is indicated by a pre-determined threshold value of dd-cfDNA of ≥0.7%, optionally wherein determining the dd-cfDNA level utilizes data from recipient genotype information.
   5. The method of any one of embodiments 1-4, wherein the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample.
   6. The method of embodiment 5, wherein the at least one mRNA transcript comprises one or more of the mRNA transcripts of Table A.
   7. The method of embodiment 6, wherein the at least one mRNA transcript comprises 2-120, 5-120, 10-120, 50-120, 80-120, 2-128, 5-128, 10-128, 50-128, 80-128, 5-50, 10-50, 50-100, or all of the mRNA transcripts of Table A.
   8. The method of any one of embodiments 1-7, wherein the sample is a blood, serum, plasma, urine, or tissue sample.
   9. The method of embodiment 8, wherein the sample is a blood sample.
   10. The method of any one of embodiments 1-9, wherein the solid organ transplant recipient is a kidney, heart, liver, pancreas, or lung transplant recipient.
   11. The method of embodiment 10, wherein the recipient is a kidney transplant recipient.
   12. The method of embodiment 11, wherein the recipient has a serum creatinine level of <2.3 mg/dL, or an increase of serum creatinine compared to baseline of no more than 10% or no more than 20%.
   13. The method of embodiment 11, wherein the recipient has a serum creatinine level of 2.3 mg/dL or higher, or an increase in serum creatinine compared to baseline of at least 10% or at least 20%.
   14. The method of any one of embodiments 1-13, wherein the method is performed at least one month, at least two months, at least three months, at least six months, or at least one year after transplantation.
   15. The method of any one of embodiments 1-14, wherein the expression level of the at least one mRNA transcript is determined by reverse transcription PCR (RT-PCR) (such as quantitative RT-PCR), hybridization to an array, or next generation sequencing.
   16. The method of any one of embodiments 1-15, wherein the dd-cfDNA level is determined by whole genome sequencing.
   17. The method of any one of embodiments 1-16, wherein determining the dd-cfDNA level comprises comparison of recipient and donor genotype information.
   18. The method of any one of embodiments 1-16, wherein the dd-cfDNA is determined without comparison to donor genotype information.
   19. The method of any one of embodiments 1-18, wherein the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects.
   20. The method of any one of embodiments 1-19, wherein the method is capable of further distinguishing likelihood of acute cellular rejection from antibody-mediated rejection, wherein the dd-cfDNA level indicates presence or absence of antibody-mediated rejection, and wherein the level of the at least one mRNA transcript indicates presence or absence of acute cellular rejection.
   21. A method of distinguishing rejection from non-rejection in a kidney transplant recipient, the method comprising
a obtaining a blood, plasma, or serum sample from the kidney transplant recipient;
b. obtaining cell-free DNA (cfDNA) and mRNA from the sample;
c. determining (i) the level of donor derived cell-free DNA (dd-cfDNA) in the cfDNA and (ii) the expression level of the at least one mRNA transcript from the sample, wherein the at least one mRNA transcript shows significantly different expression levels in kidney transplant rejection compared to kidney transplant non-rejection subjects: and
d. distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) result of a trained algorithm based on the expression level of the at least one mRNA transcript indicating rejection or non-rejection, wherein the algorithm compares the expression profile of the at least one mRNA transcript of the recipient to the expression profile of kidney transplant subjects with and without rejection.
   22. The method of embodiment 21, wherein rejection in the recipient is indicated by a pre-determined threshold value of dd-cfDNA of ≥ 0.5%, ≥ 0.6%, ≥ 0.7%, ≥ 0.8%, ≥ 0.9%, ≥ 1%, ≥ 1.2%, ≥ 1.5%, or ≥ 2%.
   23. The method of embodiment 22, wherein rejection in the recipient is indicated by a pre-determined threshold value of dd-cfDNA of ≥ 0.7%, optionally wherein determining the dd-cfDNA level utilizes data from recipient genotype information.
   24. The method of any one of embodiments 21-23, wherein the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample.
   25. The method of embodiment 24, wherein the at least one mRNA transcript comprises one or more of the mRNA transcripts of genes listed in Table A, or of at least one gene that co-expresses with or is involved in the same biological or cell signaling pathway as at least one gene listed in Table A, or of a gene a gene involved in one or more of interferon gamma signaling, CD22-mediated BCR rejection, Rho GTPase signaling, or B cell receptor signaling.
   26. The method of embodiment 25, wherein the at least one mRNA transcript comprises 2-120, 5-120, 10-120, 50-120, 80-120, 2-128, 5-128, 10-128, 50-128, 80-128, 5-50, 10-50, 50-100, or all of the mRNA transcripts of Table A.
   27. The method of any one of embodiments 21-26, wherein the recipient has a serum creatinine level of <2.3 mg/dL, or an increase of serum creatinine compared to baseline of no more than 10% or no more than 20%.
   28. The method of any one of embodiments 21-26, wherein the recipient has a serum creatinine level of 2.3 mg/dL or higher, or an increase of serum creatinine compared to baseline of no more than 10% or no more than 20%.
   29. The method of any one of embodiments 21-28, wherein the method is performed at least one month, at least two months, at least three months, at least six months, or at least one year after transplantation.
   30. The method of any one of embodiments 21-29, wherein the expression level of the at least one mRNA transcript is determined by reverse transcription PCR (RT-PCR) (such as quantitative RT-PCR), hybridization to an array, or next generation sequencing.
   31. The method of any one of embodiments 21-30, wherein the dd-cfDNA level is determined by whole genome sequencing.
   32. The method of any one of embodiments 21-31, wherein determining the dd-cfDNA level comprises comparison of recipient and donor genotype information.
   33. The method of any one of embodiments 21-31, wherein the dd-cfDNA is determined without comparison to donor genotype information.
   34. The method of any one of embodiments 21-33, wherein the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects.
   35. The method of any one of embodiments 21-34, wherein the method is capable of further distinguishing likelihood of acute cellular rejection from antibody-mediated rejection, wherein the dd-cfDNA level indicates presence or absence of antibody-mediated rejection, and wherein the level of the at least one mRNA transcript indicates presence or absence of acute cellular rejection.
   36. The method of any one of embodiments 21-35, wherein the method has a negative predictive value (NPV) of at least 85%, at least 87%, at least 88%, at least 90%, at least 92%, or at least 94% when both the level of dd-cfDNA is below the pre-determined threshold value and the result of a trained algorithm based on the expression level of the at least one mRNA transcript does not indicate rejection.
   37. The method of any one of embodiments 21-36, wherein the method has a positive predictive value (NPV) of at least 80%, at least 81%, at least 82%, at least 84%, at least 86%, at least 88%, or at least 89% when both the level of dd-cfDNA is at or above the pre-determined threshold value and the result of a trained algorithm based on the expression level of the at least one mRNA transcript indicates rejection.
   38. The method of embodiment 36 or 37, wherein determining the dd-cfDNA level utilizes data from recipient genotype information and wherein the expression level of the at least one mRNA transcript is determined by reverse-transcription PCR (RT-PCR) (such as quantitative RT-PCR).
   39. A method of treating a solid organ transplant recipient, wherein the recipient is determined to have a likelihood of rejection according to a process comprising:
a. obtaining a sample from the solid organ transplant recipient;
b. obtaining cell-free DNA (cfDNA) and mRNA from the sample;
c. determining (i) the level of donor derived cell-free DNA (dd-cfDNA) in the cfDNA and (ii) the expression level of the at least one mRNA transcript from the sample, wherein the at least one mRNA transcript shows significantly different expression levels in kidney transplant rejection compared to kidney transplant non-rejection subjects: and
d. distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) expression level of the at least one mRNA transcript or a result of an algorithm based on the expression level indicating rejection, and
wherein the recipient has received at least one immunosuppressive drug, and wherein the method comprises increasing the frequency or dosage of the at least one immunosuppressant drug, administering a further immunosuppressant drug, or administering a different immunosuppressive drug to the recipient.
   40. The method of embodiment 39, wherein the recipient is determined to have a likelihood of antibody-mediated rejection based on a level of dd-cfDNA at or above the pre-determined threshold.
   41. The method of embodiment 39, wherein the recipient is determined to have a likelihood of acute cellular rejection based on expression level of the at least one mRNA transcript or a result of an algorithm based on the expression level indicating rejection.
   42. The method of embodiment 39, wherein the recipient is determined to have a likelihood of rejection as indicated by both of (i) a level of dd-cfDNA at or above a pre-determined threshold value, and (ii) expression level of the at least one mRNA transcript or a result of an algorithm based on the expression level indicating rejection.
   43. The method of embodiment 42, wherein the method further comprises obtaining a biopsy for the recipient.
   44. The method of any one of embodiments 39-43, wherein, after increasing the frequency or dosage of the at least one immunosuppressant drug, administering a further immunosuppressant drug, or administering a different immunosuppressive drug to the recipient, the method comprises obtaining results from a repeat of the process of embodiment 39(a)-(d).
   45. The method of any one of embodiments 39-44, wherein distinguishing rejection from non-rejection comprises applying a trained algorithm to the expression level of the at least one mRNA transcript and determining a result of the algorithm, wherein the result indicates rejection or non-rejection.
   46. The method of any one of embodiments 39-45, wherein rejection in the recipient is indicated by a pre-determined threshold value of dd-cfDNA of ≥ 0.5%, ≥0.6%, ≥ 0.7%, ≥0.8%, ≥0.9%, ≥ 1%, ≥ 1.2%, ≥ 1.5%, or ≥ 2%.
   47. The method of embodiment 46, wherein rejection in the recipient is indicated by a pre-determined threshold value of dd-cfDNA of ≥0.7%, optionally wherein determining the dd-cfDNA level utilizes data from recipient genotype information.
   48. The method of any one of embodiments 39-47, wherein the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample.
   49. The method of embodiment 48, wherein the at least one mRNA transcript comprises one or more of the mRNA transcripts of Table A, or of at least one gene that co-expresses with or is involved in the same biological or cell signaling pathway as at least one gene listed in Table A, or of a gene a gene involved in one or more of interferon gamma signaling, CD22-mediated BCR rejection, Rho GTPase signaling, or B cell receptor signaling.
   50. The method of embodiment 49, wherein the at least one mRNA transcript comprises 2-120, 5-120, 10-120, 50-120, 80-120, 2-128, 5-128, 10-128, 50-128, 80-128, 5-50, 10-50, 50-100, or all of the mRNA transcripts of Table A.
   51. The method of any one of embodiments 39-50, wherein the sample is a blood, serum, plasma, urine, or tissue sample.
   52. The method of embodiment 51, wherein the sample is a blood sample.
   53. The method of any one of embodiments 39-52, wherein the solid organ transplant recipient is a kidney, heart, liver, pancreas, or lung transplant recipient.
   54. The method of embodiment 53, wherein the recipient is a kidney transplant recipient.
   55. The method of embodiment 54, wherein the recipient has a serum creatinine level of <2.3 mg/dL, or an increase of serum creatinine compared to baseline of no more than 10% or no more than 20%.
   56. The method of embodiment 54, wherein the recipient has a serum creatinine level of ≥2.3 mg/dL, or an increase of serum creatinine compared to baseline of no more than 10% or no more than 20%.
   57. The method of any one of embodiments 39-56, wherein the method is performed at least one month, at least two months, at least three months, at least six months, or at least one year after transplantation.
   58. The method of any one of embodiments 39-57, wherein the expression level of the at least one mRNA transcript is determined by reverse transcription PCR (RT-PCR) (such as quantitative RT-PCR), hybridization to an array, or next generation sequencing.
   59. The method of any one of embodiments 39-58, wherein the dd-cfDNA level is determined by whole genome sequencing.
   60. The method of any one of embodiments 39-59, wherein determining the dd-cfDNA level comprises comparison of recipient and donor genotype information.
   61. The method of any one of embodiments 39-59, wherein the dd-cfDNA is determined without comparison to donor genotype information.
   62. The method of any one of embodiments 39-61, wherein the expression level of the at least one mRNA transcript is normalized against the level of at least one reference mRNA transcript or against the level of all mRNA in the sample, wherein the at least one reference mRNA transcript does not show significantly different expression levels in transplant rejection compared to non-transplant rejection subjects.
   63. The method of any one of embodiments 1-62, wherein the pre-determined threshold value of the dd-cfDNA is determined by a multivariate regression algorithm that comprises dd-cfDNA levels and expression levels of the at least one mRNA transcript in a set of transplant recipients who received the same solid organ transplant as the recipient.
   64. A method of distinguishing rejection from non-rejection in a kidney transplant recipient with a serum creatinine level of <2.3 mg/dL or an increase of serum creatinine compared to baseline of no more than 10% or no more than 20%, or otherwise wherein kidney function tests indicate normal functioning of the transplanted kidney, the method comprising
a. obtaining a sample from the solid organ transplant recipient;
b. obtaining cell-free DNA (cfDNA);
c. determining the level of donor derived cell-free DNA (dd-cfDNA) in the cfDNA: and
d. distinguishing rejection from non-rejection in the recipient based upon a level of dd-cfDNA at or above a pre-determined threshold value.
   65. The method of embodiment 64, wherein:
a. the pre-determined threshold value of dd-cfDNA is ≥ 0.5%, ≥0.6%, ≥ 0.7%, ≥0.8%, ≥0.9%, ≥ 1%, ≥ 1.2%, ≥ 1.5%, or ≥ 2%;
b. the pre-determined threshold value of dd-cfDNA is ≥ 0.7%;
c. determining the dd-cfDNA level utilizes data from recipient genotype information;
d. the dd-cfDNA level is determined by whole genome sequencing;
e. determining the dd-cfDNA level comprises comparison of recipient and donor genotype information: and/or
f. determining the dd-cfDNA level does not comprise utilizing donor genotype information.

### EXAMPLES

### Example 1. Improved Methods of Detection of Kidney Transplant Rejection by Combining Blood Gene Expression and Cell Free DNA Analysis

### A. Design, Setting, Participants, Measurement, and Results

We performed a post-hoc analysis of simultaneous blood gene expression profile and donor derived cfDNA assays in 428 samples paired with surveillance biopsies from 208 subjects enrolled in an observational clinical trial (Clinical Trials in Organ Transplantation-08). Assay results were analyzed as binary variables and then their continuous scores combined using logistic regression. The performance of each assay alone and in combination was compared.

For diagnosing subclinical rejection, the gene expression profile demonstrated a negative predictive value (NPV) of 82%, positive predictive value (PPV) of 47%, balanced accuracy of 64%, and area under the receiver operating curve (AUROC) of 0.75. The donor derived cfDNA assay showed similar NPV (84%), PPV (56%), balanced accuracy (68%), and AUROC (0.72). When both assays were negative, NPV increased to 88%. When both assays were positive, PPV increased to 81%. Combining assays using multivariable logistic regression, AUROC was 0.81, significantly higher than gene expression profile (P<0.001) or donor derived cfDNA alone (P=0.006). Notably, when cases were separated based on rejection type, the gene expression profile was significantly better at detecting cellular rejection (AUROC 0.80 vs. 0.62, P=0.001), while the donor derived cfDNA was significantly better at detecting antibody-mediated rejection (AUROC 0.84 vs. 0.71, P=0.003).

### B. Materials and Methods

### Study Population

This post hoc analysis was performed on 428 (325 no rejection and 103 subclinical rejection) samples previously collected from 208 patients paired with surveillance kidney biopsies in the setting of stable kidney function who enrolled between 2011-2014 in the Clinical Trials in Organ Transplantation 08 (CTOT-08: NCT01289717) study. In brief, CTOT-08 was a prospective, multicenter, two-year observational study of 307 subjects. Surveillance biopsies were performed at 2 to 6, 12, and 24 months after transplant. An independent. Northwestern biorepository cohort (n=105 samples, 76 no rejection and 29 subclinical rejection) of subjects (n=85) that underwent surveillance biopsy in the first two years post-transplant (NCT01531257) was used for external validation (Table 1). The clinical and research activities being reported were consistent with the Principles of the Declaration of Istanbul as outlined in the ‘Declaration of Istanbul on Organ Trafficking and Transplant Tourism’, were subject to Institutional Review Board approval, adhered to the Declaration of Helsinki, and informed consent was obtained from all subjects.

### Materials and Methods Histologic Phenotypes and Subject Selection Criteria

The study samples were chosen to represent a screening cohort of stable patients with good kidney function. Histologic phenotypes for this analysis included subclinical rejection and no rejection. All biopsies were analyzed and scored by a blinded, central pathologist. Biopsies done for cause, in patients with serum creatinine ≥ 2.3 mg/dl, or biopsies read as having chronic fibrosis (Banff interstitial fibrosis (ci) score >1 AND tubular atrophy (ct) score >1) with or without inflammation, were excluded from the analysis (FIG. 1). The subclinical rejection clinical phenotype was defined by histology with acute rejection (≥borderline cellular rejection by Banff 2007 criteria and/or antibody mediated rejection) AND stable kidney function. Stable kidney function was specified as serum creatinine <2.3 mg/dL and <20% increase in creatinine compared with a minimum of 2 or 3 prior values. The no rejection clinical phenotype included stable kidney function AND normal histology on surveillance biopsy and was updated according to the recent changes to the Banff 2019 criteria (no evidence of rejection: Banff i=0) with t=0) or 1, g=0, ptc=0), ci=0) or 1, ct=0 or 1). It was understood that tubulitis (t2/t3) with i0 represented significant inflammation and shared traits with newly defined borderline changes. Therefore, tubulitis (t2/t3) was classified with i0 as borderline changes in this study. Because donor specific HLA antibody (DSA) information was not available for the majority of samples, histologic criteria for diagnosing antibody mediated rejection in samples missing paired DSA information were used. The biopsy was classified as antibody mediated rejection if 2 histologic criteria of acute antibody mediated rejection according to 2019 Banff classification were present. If a specimen met one out of 2 histologic criteria of acute antibody mediated rejection, it was classified as “suspicious antibody mediated rejection.” Gene expression profile and donor derived cfDNA performance were analyzed by including the “suspicious antibody mediated rejection” in the antibody mediated rejection group, to capture biopsies with even low levels of microvascular inflammation. For example, a sample with ptc=1 was considered “suspicious antibody mediated rejection.”

### Gene Expression Profile

Blood samples for the gene expression profile assay were drawn directly into PAXgene (BD BioSciences, San Jose, CA) tubes at the time of surveillance biopsy. The samples were processed as using Affymetrix HT HG-U133+PM Array Plates on the Gene Titan MC instrument (Thermo Fisher Scientific, Waltham, MA) (deposited as GEO Accession No. GSE107509) according to manufacturer's instructions. The gene expression profiles were analyzed with the TruGraf® algorithm—a DNA microarray-based gene expression algorithm analyzing differential expression of 120 genes- and assigned a result of either TX or not-TX. Gene expression profile results were provided as a probability score normalized on a 0-100 scale. The TruGraf® assay (Eurofins—Transplant Genomics, Framingham, MA) has a previously defined probability threshold of 50 to differentiate the TX (normal, no rejection) from the not-TX phenotype (including subclinical rejection). The 120 genes used for the TruGraf® assay are described in Table A below. The human genes listed in Table A are identified by their full name and gene symbols, as well as by the Probe Set ID provided for each of the genes in the Affymetrix HG-U133 Plus PM microarray (Array Name “HT_HG-U133_Plus_PM”).

TABLE A
Probes and corresponding genes assessed in the TruGraf ® gene expression assay.

| | | Gene | | |
|---|---|---|---|---|
| # | Probe Set Id | Symbol | Gene Title | Array Name |
| 1 | 1553856_PM_s_at | P2RY10 | purinergic receptor P2Y, G-protein coupled, 10 | HT_HG-U133_Plus_PM |
| 2 | 1554608_PM_at | TGOLN2 | trans-golgi network protein 2 | HT_HG-U133_Plus_PM |
| 3 | 1555730_PM_a_at | CFL1 | cofilin 1 (non-muscle) | HT_HG-U133_Plus_PM |
| 4 | 1555812_PM_a_at | ARHGDIB | Rho GDP dissociation inhibitor (GDI) beta | HT_HG-U133_Plus_PM |
| 5 | 1556033_PM_at | LINC01138 | long intergenic non-protein coding RNA 1138 | HT_HG-U133_Plus_PM |
| 6 | 1557116_PM_at | APOL6 | apolipoprotein L, 6 | HT_HG-U133_Plus_PM |
| 7 | 1561058_PM_at | — | Homo sapiens cDNA clone IMAGE:5278570. | HT_HG-U133_Plus_PM |
| 8 | 1562505_PM_at | — | gb:BC035700.1/DB_XREF = gi:23272849/ | HT_HG-U133_Plus_PM |
| | | | TID = Hs2.337138.1/CNT = 2/FEA = mRNA/ | |
| | | | TIER = ConsEnd/STK = 0/UG = Hs.337138/ | |
| | | | UG_TITLE = Homo sapiens, clone | |
| | | | IMAGE:5550275, mRNA/DEF = Homo | |
| | | | sapiens, clone IMAGE:5550275, mRNA. | |
| 9 | 1565913_PM_at | — | Homo sapiens full length insert cDNA clone YR04D03. | HT_HG-U133_Plus_PM |
| 10 | 1566129_PM_at | LIMS1 | LIM and senescent cell antigen-like domains 1 | HT_HG-U133_Plus_PM |
| 11 | 1570264_PM_at | — | Homo sapiens, clone IMAGE:4337699, mRNA. | HT_HG-U133_Plus_PM |
| 12 | 200041_PM_s_at | ATP6V1G2- | ATP6V1G2-DDX39B readthrough (NMD | HT_HG-U133_Plus_PM |
| | | DDX39B /// | candidate) /// DEAD (Asp-Glu-Ala-Asp) box | |
| | | DDX39B | polypeptide 39B | |
| 13 | 200623_PM_s_at | CALM2 /// | calmodulin 2 (phosphorylase kinase, delta) /// | HT_HG-U133_Plus_PM |
| | | CALM3 | calmodulin 3 (phosphorylase kinase, | |
| | | | delta) | |
| 14 | 200634_PM_at | PFN1 | profilin 1 | HT_HG-U133_Plus_PM |
| 15 | 200745_PM_s_at | GNB1 | guanine nucleotide binding protein (G | HT_HG-U133_Plus_PM |
| | | | protein), beta polypeptide 1 | |
| 16 | 200885_PM_at | RHOC | ras homolog family member C | HT_HG-U133_Plus_PM |
| 17 | 201236_PM_s_at | BTG2 | BTG family, member 2 | HT_HG-U133_Plus_PM |
| 18 | 201251_PM_at | PKM | pyruvate kinase, muscle | HT_HG-U133_Plus_PM |
| 19 | 201537_PM_s_at | DUSP3 | dual specificity phosphatase 3 | HT_HG-U133_Plus_PM |
| 20 | 201612_PM_at | ALDH9A1 | aldehyde dehydrogenase 9 family, member A1 | HT_HG-U133_Plus_PM |
| 21 | 202080_PM_s_at | TRAK1 | trafficking protein, kinesin binding 1 | HT_HG-U133_Plus_PM |
| 22 | 202333_PM_s_at | UBE2B | ubiquitin conjugating enzyme E2B | HT_HG-U133_Plus_PM |
| 23 | 202366_PM_at | ACADS | acyl-CoA dehydrogenase, C-2 to C-3 short | HT_HG-U133_Plus_PM |
| | | | chain | |
| 24 | 203273_PM_s_at | TUSC2 | tumor suppressor candidate 2 | HT_HG-U133_Plus_PM |
| 25 | 203921_PM_at | CHST2 | carbohydrate (N-acetylglucosamine-6-0) | HT_HG-U133_Plus_PM |
| | | | sulfotransferase 2 | |
| 26 | 204516_PM_at | ATXN7 | ataxin 7 | HT_HG-U133_Plus_PM |
| 27 | 205297_PM_s_at | CD79B | CD79b molecule, immunoglobulin- | HT_HG-U133_Plus_PM |
| | | | associated beta | |
| 28 | 205495_PM_s_at | GNLY | granulysin | HT_HG-U133_Plus_PM |
| 29 | 205603_PM_s_at | DIAPH2 | diaphanous-related formin 2 | HT_HG-U133_Plus_PM |
| 30 | 205905_PM_s_at | MICA /// | MHC class I polypeptide-related sequence A /// | HT_HG-U133_Plus_PM |
| | | MICB | MHC class I polypeptide-related | |
| | | | sequence B | |
| 31 | 206652_PM_at | ZMYM5 | zinc finger, MYM-type 5 | HT_HG-U133_Plus_PM |
| 32 | 207194_PM_s_at | ICAM4 | intercellular adhesion molecule 4 | HT_HG-U133_Plus_PM |
| | | | (Landsteiner-Wiener blood group) | |
| 33 | 208174_PM_x_at | ZRSR2 | zinc finger (CCCH type), RNA binding motif | HT_HG-U133_Plus_PM |
| | | | and serine/arginine rich 2 | |
| 34 | 208784_PM_s_at | KLHDC3 | kelch domain containing 3 | HT_HG-U133_Plus_PM |
| 35 | 208997_PM_s_at | UCP2 | uncoupling protein 2 (mitochondrial, proton | HT_HG-U133_Plus_PM |
| | | | carrier) | |
| 36 | 209199_PM_s_at | MEF2C | myocyte enhancer factor 2C | HT_HG-U133_Plus_PM |
| 37 | 209304_PM_x_at | GADD45B | growth arrest and DNA-damage-inducible, | HT_HG-U133_Plus_PM |
| | | | beta | |
| 38 | 209306_PM_s_at | SWAP70 | SWAP switching B-cell complex 70 kDa | HT_HG-U133_Plus_PM |
| | | | subunit | |
| 39 | 210057_PM_at | SMG1 | SMG1 phosphatidylinositol 3-kinase-related | HT_HG-U133_Plus_PM |
| | | | kinase | |
| 40 | 210125_PM_s_at | BANF1 | barrier to autointegration factor 1 | HT_HG-U133_Plus_PM |
| 41 | 210253_PM_at | HTATIP2 | HIV-1 Tat interactive protein 2 | HT_HG-U133_Plus_PM |
| 42 | 210356_PM_x_at | MS4A1 | membrane-spanning 4-domains, subfamily | HT_HG-U133_Plus_PM |
| | | | A, member 1 | |
| 43 | 210985_PM_s_at | SP100 | SP100 nuclear antigen | HT_HG-U133_Plus_PM |
| 44 | 210996_PM_s_at | YWHAE | tyrosine 3-monooxygenase/tryptophan 5- | HT_HG-U133_Plus_PM |
| | | | monooxygenase activation protein, epsilon | |
| 45 | 210999_PM_s_at | GRB10 | growth factor receptor bound protein 10 | HT_HG-U133_Plus_PM |
| 46 | 211207_PM_s_at | ACSL6 | acyl-CoA synthetase long-chain family | HT_HG-U133_Plus_PM |
| | | | member 6 | |
| 47 | 212099_PM_at | RHOB | ras homolog family member B | HT_HG-U133_Plus_PM |
| 48 | 212386_PM_at | TCF4 | transcription factor 4 | HT_HG-U133_Plus_PM |
| 49 | 212467_PM_at | DNAJC13 | DnaJ (Hsp40) homolog, subfamily C, | HT_HG-U133_Plus_PM |
| | | | member 13 | |
| 50 | 212762_PM_s_at | TCF7L2 | transcription factor 7-like 2 (T-cell specific, | HT_HG-U133_Plus_PM |
| | | | HMG-box) | |
| | 213286_PM_at | ZFR | zinc finger RNA binding protein | HT_HG-U133_Plus_PM |
| 52 | 214511_PM_x_at | FCGR1B | Fc fragment of IgG, high affinity Ib, receptor | HT_HG-U133_Plus_PM |
| | | | (CD64) | |
| 53 | 214669_PM_x_at | IGK /// | immunoglobulin kappa locus /// | HT_HG-U133_Plus_PM |
| | | IGKC /// | immunoglobulin kappa constant /// | |
| | | IGKV1-5 /// | immunoglobulin kappa variable 1-5 /// | |
| | | IGKV3-20 /// | immunoglobulin kappa variable 3-20 /// | |
| | | IGKV3D-20 | immunoglobulin kappa variable 3D-20 | |
| 54 | 214907_PM_at | CEACAM21 | carcinoembryonic antigen-related cell | HT_HG-U133_Plus_PM |
| | | | adhesion molecule 21 | |
| 55 | 216069_PM_at | PRMT2 | protein arginine methyltransferase 2 | HT_HG-U133_Plus_PM |
| 56 | 216950_PM_s_at | FCGR1A /// | Fc fragment of IgG, high affinity Ia, receptor | HT_HG-U133_Plus_PM |
| | | FCGR1C | (CD64) /// Fc fragment of IgG, high affinity | |
| | | | Ic, receptor (CD64), pseudogene | |
| 57 | 217418_PM_x_at | MS4A1 | membrane-spanning 4-domains, subfamily | HT_HG-U133_Plus_PM |
| | | | A, member 1 | |
| 58 | 217436_PM_x_at | HLA-J | major histocompatibility complex, class I, J | HT_HG-U133_Plus_PM |
| | | | (pseudogene) | |
| 59 | 217979_PM_at | TSPAN13 | tetraspanin 13 | HT_HG-U133_Plus_PM |
| 60 | 217991_PM_x_at | SSBP3 | single stranded DNA binding protein 3 | HT_HG-U133_Plus_PM |
| 61 | 218438_PM_s_at | MED28 | mediator complex subunit 28 | HT_HG-U133_Plus_PM |
| 62 | 218527_PM_at | APTX | aprataxin | HT_HG-U133_Plus_PM |
| 63 | 219100_PM_at | OBFC1 | oligonucleotide/oligosaccharide-binding fold | HT_HG-U133_Plus_PM |
| | | | containing 1 | |
| 64 | 219191_PM_s_at | BIN2 | bridging integrator 2 | HT_HG-U133_Plus_PM |
| 65 | 219233_PM_s_at | GSDMB | gasdermin B | HT_HG-U133_Plus_PM |
| 66 | 219471_PM_at | KIAA0226L | KIAA0226-like | HT_HG-U133_Plus_PM |
| 67 | 219938_PM_s_at | PSTPIP2 | proline-serine-threonine phosphatase | HT_HG-U133_Plus_PM |
| | | | interacting protein 2 | |
| 68 | 219966_PM_x_at | BANP | BTG3 associated nuclear protein | HT_HG-U133_Plus_PM |
| 69 | 221013_PM_s_at | APOL2 | apolipoprotein L, 2 | HT_HG-U133_Plus_PM |
| 70 | 221508_PM_at | TAOK3 | TAO kinase 3 | HT_HG-U133_Plus_PM |
| 71 | 222471_PM_s_at | KCMF1 | potassium channel modulatory factor 1 | HT_HG-U133_Plus_PM |
| 72 | 222582_PM_at | PRKAG2 | protein kinase, AMP-activated, gamma 2 | HT_HG-U133_Plus_PM |
| | | | non-catalytic subunit | |
| 73 | 222799_PM_at | WDR91 | WD repeat domain 91 | HT_HG-U133_Plus_PM |
| 74 | 222891_PM_s_at | BCL11A | B-cell CLL/lymphoma 11A (zinc finger | HT_HG-U133_Plus_PM |
| | | | protein) | |
| 75 | 222996_PM_s_at | CXXC5 | CXXC finger protein 5 | HT_HG-U133_Plus_PM |
| 76 | 223465_PM_at | COL4A3BP | collagen, type IV, alpha 3 (Goodpasture | HT_HG-U133_Plus_PM |
| | | | antigen) binding protein | |
| 77 | 223950_PM_s_at | FLYWCH1 | FLYWCH-type zinc finger 1 | HT_HG-U133_Plus_PM |
| 78 | 224516_PM_s_at | CXXC5 | CXXC finger protein 5 | HT_HG-U133_Plus_PM |
| 79 | 224549_PM_x_at | — | metastasis associated lung adenocarcinoma | HT_HG-U133_Plus_PM |
| | | | transcript 1 (non-protein coding) | |
| 80 | 224559_PM_at | MALAT1 | metastasis associated lung adenocarcinoma | HT_HG-U133_Plus_PM |
| | | | transcript 1 (non-protein coding) | |
| 81 | 224767_PM_at | LOC100506548 /// | uncharacterized LOC100506548 /// | HT_HG-U133_Plus_PM |
| | | RPL37 | ribosomal protein L37 | |
| 82 | 224840_PM_at | FKBP5 | FK506 binding protein 5 | HT_HG-U133_Plus_PM |
| 83 | 225012_PM_at | HDLBP | high density lipoprotein binding protein | HT_HG-U133_Plus_PM |
| 84 | 225108_PM_at | AGPS | alkylglycerone phosphate synthase | HT_HG-U133_Plus_PM |
| 85 | 225232_PM_at | MTMR12 | myotubularin related protein 12 | HT_HG-U133_Plus_PM |
| 86 | 225294_PM_s_at | TRAPPC1 | trafficking protein particle complex 1 | HT_HG-U133_Plus_PM |
| 87 | 225870_PM_s_at | TRAPPC5 | trafficking protein particle complex 5 | HT_HG-U133_Plus_PM |
| 88 | 225933_PM_at | CCDC137 | coiled-coil domain containing 137 | HT_HG-U133_Plus_PM |
| 89 | 226518_PM_at | KCTD10 | potassium channel tetramerization domain | HT_HG-U133_Plus_PM |
| | | | containing 10 | |
| 90 | 227052_PM_at | SMIM14 | small integral membrane protein 14 | HT_HG-U133_Plus_PM |
| 91 | 227410_PM_at | FAM43A | family with sequence similarity 43, member A | HT_HG-U133_Plus_PM |
| 92 | 227458_PM_at | CD274 | CD274 molecule | HT_HG-U133_Plus_PM |
| 93 | 227787_PM_s_at | MED30 | mediator complex subunit 30 | HT_HG-U133_Plus_PM |
| 94 | 228928_PM_x_at | BANP | BTG3 associated nuclear protein | HT_HG-U133_Plus_PM |
| 95 | 229187_PM_at | LOC283788 | FSHD region gene 1 pseudogene | HT_HG-U133_Plus_PM |
| 96 | 231035_PM_s_at | OTUD1 | OTU deubiquitinase 1 | HT_HG-U133_Plus_PM |
| 97 | 232340_PM_at | MIATNB | MIAT neighbor (non-protein coding) | HT_HG-U133_Plus_PM |
| 98 | 232375_PM_at | — | gb:AI539443/DB_XREF = gi:4453578/ | HT_HG-U133_Plus_PM |
| | | | DB_XREF = te51e11.x1/ | |
| | | | CLONE = IMAGE:2090252/FEA = mRNA/ | |
| | | | CNT = 10/TID = Hs.137447.0/TIER = ConsEnd/ | |
| | | | STK = 3/UG = Hs.137447/UG_TITLE = Homo | |
| | | | sapiens cDNA FLJ12169 fis, clone | |
| | | | MAMMA1000643 | |
| 99 | 232405_PM_at | — | Homo sapiens cDNA: FLJ22832 fis, clone | HT_HG-U133_Plus_PM |
| | | | KAIA4195 | |
| 100 | 232420_PM_x_at | MAN1B1- | MAN1B1 antisense RNA 1 (head to head) | HT_HG-U133_Plus_PM |
| | | AS1 | | |
| 101 | 232864_PM_s_at | AFF4 | AF4/FMR2 family, member 4 | HT_HG-U133_Plus_PM |
| 102 | 233186_PM_s_at | BANP | BTG3 associated nuclear protein | HT_HG-U133_Plus_PM |
| 103 | 233309_PM_at | — | Homo sapiens cDNA FUJ11759 fis, clone | HT_HG-U133_Plus_PM |
| | | | HEMBA1005616 | |
| 104 | 235461_PM_at | TET2 | tet methylcytosine dioxygenase 2 | HT_HG-U133_Plus_PM |
| 105 | 235533_PM_at | COX19 | COX19 cytochrome c oxidase assembly | HT_HG-U133_Plus_PM |
| | | | factor | |
| 106 | 235645_PM_at | ESCO1 | establishment of sister chromatid cohesion | HT_HG-U133_Plus_PM |
| | | | N-acetyltransferase 1 | |
| 107 | 236298_PM_at | PDSS1 | prenyl (decaprenyl) diphosphate synthase, | HT_HG-U133_Plus_PM |
| | | | subunit 1 | |
| 108 | 239294_PM_at | PIK3CG | phosphatidylinositol-4,5-bisphosphate 3- | HT_HG-U133_Plus_PM |
| | | | kinase, catalytic subunit gamma | |
| 109 | 240008_PM_at | — | gb:A1955765/DB_XREF = gi:5748075/ | HT_HG-U133_Plus_PM |
| | | | DB_XREF = wt59c08.x1/ | |
| | | | CLONE = IMAGE:2511758/FEA = EST/CNT = 7/ | |
| | | | TID = Hs. 146907.0/TIER = ConsEnd/STK = 1/ | |
| | | | UG = Hs.146907/UG_TITLE = ESTs | |
| 110 | 242014_PM_at | — | gb:AI825538/DB_XREF = gi:5446209/ | HT_HG-U133_Plus_PM |
| | | | DB_XREF = wb18h06.x1/ | |
| | | | CLONE = IMAGE:2306075/FEA = EST/CNT = 3/ | |
| | | | TID = Hs. 187534.0/TIER = ConsEnd/STK = 3/ | |
| | | | UG = Hs.187534/UG_TITLE = ESTs | |
| 111 | 242374_PM_at | — | nx92b05.s1 Homo sapiens cDNA/ | HT_HG-U133_Plus_PM |
| | | | clone = IMAGE-1269681/gb = AA747563/ | |
| | | | gi = 2787521/ug = Hs.131799/len = 325 | |
| 112 | 242751_PM_at | — | qu42g07.x1 Homo sapiens cDNA, 3′ end/ | HT_HG-U133_Plus_PM |
| | | | clone = IMAGE-1967484/clone_end = 3′/ | |
| | | | gb = AI281464/gi = 3919697/ug = Hs.38038/ | |
| | | | len = 387 | |
| 113 | 242918_PM_at | NASP | nuclear autoantigenic sperm protein | HT_HG-U133_Plus_PM |
| | | | (histone-binding) | |
| 114 | 243417_PM_at | ZADH2 | zinc binding alcohol dehydrogenase domain | HT_HG-U133_Plus_PM |
| | | | containing 2 | |
| 115 | 243981_PM_at | STK4 | serine/threonine kinase 4 | HT_HG-U133_Plus_PM |
| 116 | 244433_PM_at | — | accn = NULL class = lincRNA name = Human | HT_HG-U133_Plus_PM |
| | | | lincRNA ref = Scripture Reconstruction | |
| | | | LincRNAs By Luo transcriptld = linc_luo_1183 | |
| | | | cpcScore = −1.3227000 cnci = −0.4318137 | |
| 117 | 44790_PM_s_at | KIAA0226L | KIAA0226-like | HT_HG-U133_Plus_PM |
| 118 | 50314_PM_i_at | C20orf27 | chromosome 20 open reading frame 27 | HT_HG-U133_Plus_PM |
| 119 | 54632_PM_at | THADA | thyroid adenoma associated | HT_HG-U133_Plus_PM |
| 120 | 59644_PM_at | BMP2K | BMP2 inducible kinase | HT_HG-U133_Plus_PM |
| 121 | | DIP2C | disco interacting protein 2 homolog C | |
| 122 | | ENOSF1 | enolase superfamily member 1 | |
| 123 | | FBXO21 | F-box protein 21 | |
| 124 | | KCTD6 | potassium channel tetramerization domain | |
| | | | containing 6 | |
| 125 | | PDXDC1 | pyridoxal dependent decarboxylase domain | |
| | | | containing 1 | |
| 126 | | REXO2 | RNA exonuclease 2 | |
| 127 | | HLA-E | major histocompatibility complex, class I, E | |
| 128 | | RAB31 | RAB31, member RAS oncogene family | |

### Donor-Derived Cell-Free DNA

Blood for donor derived cfDNA analysis was also drawn at the time of surveillance biopsy in plasma separation tubes (BD Vacutainer® PPT™ Plasma Preparation Tube, BD BioSciences, San Jose, CA). Next generation sequencing data were mapped to a reference genome and, along with recipient genotype data (see below), analyzed for percentage donor derived cfDNA by a bioinformatics pipeline licensed from Stanford University essentially as described in WO2018187226A1, which is incorporated in its entirety by reference herein.

Recipient genotyping was performed on PBMC samples. The donor derived cfDNA results were provided as a percentage of the donor-derived fraction as compared to total cfDNA by using panels of single nucleotide polymorphisms (SNPs) (approximately 70,000 SNPs) to differentiate between donor and recipient cfDNA without requiring knowledge of donor genotypes. The Viracor TRAC® assay (Eurofins—Viracor, Lenexa, KS, USA) reports the fraction of donor derived cfDNA as a percentage with >0.7% being considered positive. Results of the assay with additional thresholds to allow comparison with other commercial donor derived cfDNA assays were also reported.

### Statistical Analysis

Demographic characteristics were compared among the three groups with different histological phenotypes using one-way analysis of variance for continuous variables and chi-square test for nominal variables, respectively. Biopsies, gene expression profile, and donor derived cfDNA results were treated as binary outcomes for performance analyses. Sensitivity, specificity, PPV, NPV, Area Under the Receiver Operating Curve (AUROC), accuracy, and balanced accuracy were calculated. Bootstrapping with 10,000 iterations was used to calculate a 95% CI for each performance metric. To assess the performance of combined gene expression profile and donor derived cfDNA continuous scores, multivariable logistic regression was performed using the continuous scores of both assays. External validation was performed using an independent Northwestern biorepository cohort. We considered p-value <0.05 as statistically significant in a two-tailed test. All statistical analyses were performed using R version 4.0.0 via RStudio.

### C. Results

428 blood samples were analyzed from 208 unique subjects who had surveillance biopsies paired with gene expression profiles and donor derived cfDNA. Of 208 subjects, 11% (n=22) had only subclinical rejection (i.e., no normal biopsies), 59% (n=123) had no rejection only (e.g., no biopsies with subclinical rejection), and 30% (n=63) with either subclinical rejection or no rejection (e.g., ≥1 episode of subclinical rejection during the study period) based on histological phenotypes (Table 1). There was no significant difference in patient-level demographics between the three groups except for race, desensitization therapy and steroid use as a maintenance therapy. Of 428 samples, 76% (n=325) and 24% (n=103) were classified no rejection and subclinical rejection, respectively, by histologic phenotypes. The 103 subclinical rejection biopsies consisted of borderline 32% (n=33), Banff ≥1A 5% (n=5), borderline+suspicious antibody mediated rejection 13% (n=13), Banff≥1A+suspicious antibody mediated rejection 4% (n=4), suspicious antibody mediated rejection 21% (n=22), antibody mediated rejection only 20% (n=20), antibody mediated rejection with ≥borderline 6% (n=6). Of 65 antibody mediated rejection biopsy samples, 23 (35%) had cellular rejection components and 42 cases (65%) had antibody mediated rejection only. Subclinical rejection occurred almost evenly at the different biopsy time points in the study period. Of 103 subclinical rejection cases, 21%, 32%, 31%, and 16% were identified at 3-6, 12, 24 months, and intensive monitoring period or other time, respectively (Table 2).

TABLE 1
Characteristics of study participants and kidney donors
Clinical Trials in Organ Transplantation Northwestern University Validation

| | 08 cohort | | | Cohort | | |
|---|---|---|---|---|---|---|
| | | | Mixed | | | Mixed |
| | | | subclinical | | | subclinical |
| | Only | No | rejection | Only | No | rejection |
| | Subclinical | subclinical | and No | subclinical | subclinical | and No |
| | rejection | rejection | Rejection | rejection | rejection | Rejection |
| Demographics | (n = 22) | (n = 123) | (n = 63) | (n = 21) | (n = 59) | (n = 5) |
| Kidney Donors | | | | | | |
| Deceased donor, n (%) | 16 (73) | 46 (37) | 22 (25) | 11 (52) | 20 (34) | 2 (40) |
| Donor age mean (±SD) | 35 (16) | 41 (13) | 41 (13) | 39 (14) | 36 (13) | 46 (17) |
| Donor sex, female, n, (%) | 10 (46) | 62 (50) | 29 (46) | 9 (43) | 30 (51) | 3 (60) |
| Donor race, n (%) | | | | | | |
| White | 17 (77) | 82 (67) | 50 (80) | 15 (57) | 32 (54) | 3 (60) |
| Black or African American | 2 (9) | 21 (17) | 2 (3) | 2 (10) | 11 (19) | 0 (0) |
| American Indian or | 0 (0) | 2 (2) | 0 (0) | 0 (0) | 0 (0) | 0 (0) |
| Alaska Native | | | | | | |
| Native Hawaiian/other | 0 (0) | 0 (0) | 1 (2) | 0 (0) | 0 (0) | 0 (0) |
| Pacific Islander | | | | | | |
| Asian | 1 (5) | 4 (3) | 2 (3) | 1 (5) | 3 (5) | 0 (0) |
| Unknown | 2 (9) | 14 (11) | 8 (13) | 3 (14) | 0 (0) | 0 (0) |
| Donor ethnicity (%) | | | | | | |
| Not Hispanic or Latino | 16 (73) | 91 (74) | 47 (75) | 8 (38) | 22 (37) | 2 (40) |
| Hispanic or Latino | 3 (14) | 17 (14) | 8 (13) | 4 (19) | 1 (2) | 0 (0.0) |
| Unknown | 3 (14) | 15 (12) | 8 (13) | 9 (43) | 36 (61) | 3 (60) |
| Kidney recipients | | | | | | |
| Recipients age mean (±SD) | 48 (16) | 51 (15) | 53 (14) | 49 (12) | 50 (13) | 58 (15) |
| Recipients sex, female, n (%) | 6 (27) | 44 (36) | 21 (33) | 10 (48) | 25 (42) | 3 (60) |
| Recipients race, n (%) | | | | | | |
| White | 14 (64) | 73 (59) | 43 (68) | 12 (57) | 31 (53) | 2 (40) |
| Black or African American | 6 (27) | 29 (24) | 7 (11) | 5 (24) | 10 (17) | 0 (0) |
| American Indian or | 2 (9) | 0 (0) | 2 (3) | 0 (0) | 1 (2) | 0 (0) |
| Alaska Native | | | | | | |
| Native Hawaiian/Other | 0 (0) | 1 (1) | 1 (2) | 0 (0) | 0 (0) | 0 (0) |
| Pacific Islander | | | | | | |
| Asian | 0 (0) | 10 (8) | 1 (2) | 1 (5) | 5 (9) | 0 (0) |
| Unknown | 0 (0) | 9 (7) | 8 (13) | 2 (10) | 4 (7) | 0 (0) |
| Recipients Ethnicity, n (%) | | | | | | |
| Not Hispanic or Latino | 19 (86) | 101 (82) | 47 (75) | 17 (81) | 47 (80) | 2 (40) |
| Hispanic or Latino | 2 (9) | 18 (15) | 11 (18) | 2 (10) | 3 (5) | 0 (0) |
| Unknown | 1 (5) | 4 (3) | 5 (8) | 2 (10) | 3 (5) | 0 (0) |
| Recipients-primary reason | | | | | | |
| for kidney failure, n (%) | | | | | | |
| Cystic (includes PKD) | 0 (0) | 12 (10) | 10 (16) | 2 (10) | 7 (12) | 1 (20) |
| Diabetes mellitus | 5 (23) | 26 (21) | 11 (18) | 2 (10) | 10 (17) | 2 (40) |
| Glomerulonephritis | 6 (27) | 36 (29) | 14 (22) | 8 (38) | 22 (37) | 1 (20) |
| Hypertension | 2 (9) | 25 (20) | 11 (18) | 6 (29) | 15 (25) | 1 (20) |
| Other | 9 (41) | 24 (20) | 17 (27) | 3 (14) | 5 (9) | 0 (0) |
| Recipient PRA at transplant | | | | | | |
| PRA class I, | 0 [0, 9] | 0 [0, 0] | 0 [0, 0] | 11 [0, 51] | 4 [0, 15] | 4 [0, 20] |
| % (median [IQR]) | | | | | | |
| PRA class II, | 0 [0, 6] | 0 [0, 0] | 0 [0, 0] | 2 [0, 75] | 0 [0, 18] | 0 [0, 4] |
| % (median [IQR]) | | | | | | |
| CPRA, % (median [IQR]) | 18 [0, 97] | 9 [0, 54] | 0 [0, 64] | 12 [0, 54] | 0 [0, 0] | 0 [0, 0] |
| Induction therapy, n (%) | | | | | | |
| Basiliximab | 4 (18) | 21 (17) | 16 (25) | 4 (19) | 8 (14) | 1 (20) |
| Alemtuzumab | 9 (41) | 59 (48) | 33 (52) | 17 (81) | 51 (86) | 5 (100) |
| Anti-thymocyte globulin | 10 (46) | 37 (30) | 13 (21) | 0 (0) | 0 (0) | 0 (0) |
| Steroid | 18 (82) | 97 (79) | 52 (83) | 21 (100) | 59 (100) | 5 (100) |
| IVIG | 1 (5) | 3 (2) | 0 (0) | 0 (0) | 0 (0) | 0 (0) |
| Rituximab | 0 (0) | 0 (0) | 0 (0) | 5 (24) | 6 (10) | 0 (0) |
| Received desensitization | 0 (0) | 5 (4) | 8 (13) | 5 (24) | 7 (12) | 0 (0) |
| therapy, n (%) | | | | | | |
| Maintenance therapy, n (%) | | | | | | |
| Steroid | 18 (82) | 56 (46) | 42 (67) | 7 (33) | 9 (15) | 1 (20) |
| Tacrolimus | 22 (100) | 122 (99) | 63 (100) | 16 (76) | 51 (86) | 5 (100) |
| Cyclosporine | 2 (9) | 5 (4) | 4 (6) | 1 (5) | 0 (0) | 0 (0) |
| Azathioprine | 0 (0) | 0 (0) | 0 (0) | 1 (5) | 0 (0) | 0 (0) |
| Mycophenolate | 22 (100) | 121 (98) | 62 (98) | 16 (76) | 47 (80) | 5 (100) |
| Sirollmus | 1 (5) | 8 (7) | 7 (11) | 2 (10) | 1 (2) | 0 (0) |
| Leflunomide | 0 (0) | 1 (1) | 2 (3) | 0 (0) | 0 (0) | 0 (0) |
| Belatacept | 0 (0) | 1 (1) | 0 (0) | 0 (0) | 0 (0) | 0 (0) |
| Unknown | 0 (0) | 0 (0) | 0 (0) | 2 (10) | 8 (14) | 0 (0) |

In Table 1, results are shown for 208 subjects with stable kidney function underwent surveillance biopsies. 22 (11%), 123 (59%), and 63 (30%) had only subclinical rejection, no rejection, and mixed either no rejection or subclinical rejection (e.g., ≥1 episode of subclinical rejection during the study period), respectively. The median PRA (Panel Reactive Antibodies) and cPRA (Calculated Panel Reactive Antibodies) are reported in Table 1 since they were not normally distributed.

In the Northwestern validation cohort, 85 subjects with stable kidney function underwent surveillance biopsies. 21 (25%), 59 (69%), and 5 (6%) had only subclinical rejection, no rejection, and mixed either no rejection or subclinical rejection (e.g., ≥ 1 episode of subclinical rejection during the study period), respectively.

TABLE 2
Subclinical acute rejection types, timing of rejections, and number positive by each assay

| Number and timing | | | | |
|---|---|---|---|---|
| of rejection/type of | 3-6 months | 12 months | 24 months | IM or SR |
| rejection | 21% | 32% | 31% | 16% |
| (n = 103) | (n = 22) | (n = 33) | (n = 32) | (n = 16) |
| Acute cellular | 10 | 10 | 12 | 6 |
| rejection Banff | (6 gene expression | (5 gene expression | (7 gene expression | (1 gene expression |
| Borderline or ≥1A | profile, 0 cfDNA) | profile, 0 cfDNA) | profile, 3 cfDNA) | profile, 1 cfDNA) |
| 37% (n = 38) | | | | |
| Mixed acute cellular | 5 | 10 | 3 | 5 |
| rejection + antibody | (1 gene expression | (3 gene expression | (2 gene expression | (4 gene expression |
| mediated rejection | profile, 3 cfDNA) | profile, 6 cfDNA) | profile, 1 cfDNA) | profile, 3 cfDNA) |
| 22% (n = 23) | | | | |
| Suspicious antibody | 7 | 13 | 17 | 5 |
| mediated rejection + | (2 gene expression | (5 gene expression | (4 gene expression | (4 gene expression |
| antibody mediated | profile, 3 cfDNA) | profile, 10 cfDNA) | profile, 13 cfDNA) | profile, 5 cfDNA) |
| rejection 41% (n = 42) | | | | |

In Table 2, results are shown for a total of 103 subclinical rejection cases that were identified by histologic evaluation. The acute cellular rejection cases consisted of borderline (n=33) and Banff≥1A (n=5). The mixed group included borderline+suspicious antibody mediated rejection (n=13), Banff≤A+suspicious antibody mediated rejection (n=4), and antibody mediated rejection with ≥borderline (n=6). There were 22 cases of suspicious antibody mediated rejection and 20 of antibody mediated rejection in the suspicious antibody mediated rejection+antibody mediated rejection group. Columns depict the timing post-transplant when rejection episodes occurred (with % of total rejections and number in parentheses). Each square demonstrates the total number of rejection cases based on clinical/histologic phenotype followed by the number of true positive tests detected by each assay in parentheses. In Table 2, cfDNA denotes donor derived cell free DNA assay; IM denotes Intense Monitoring Visit; and SR denotes suspected rejection visit (but found to have stable function meeting definition of subclinical acute rejection).

Diagnostic Performance of Gene Expression, Donor Derived cfDNA, and the Combination of the Two Tests-Gene Expression Profile Performance

In 103 subclinical rejection cases, the gene expression profile was positive (not-TX) in 43% (n=44) and negative (TX) in 57% (n=59). Of the 325 normal biopsy cases, the gene expression profile was negative in 85% (n=275) and positive in 15% (n=50). Full performance metrics are listed in Table 3.

TABLE 3
Summary of diagnostic metrics to detect subclinical acute rejection.

| | Gene | Donor | Positive = Gene | Positive = Gene | |
|---|---|---|---|---|---|
| | expression | derived | expression profile + OR | expression profile + AND | Logistic |
| | profile | cfDNA | donor derived | donor derived | Regression |
| Diagnostic | alone | alone | cfDNA+ | cfDNA+ | at 0.35 |
| performance | (95% CI) | (95% CI) | (95% CI) | (95% CI) | cutoff |
| Sensitivity | 0.43 | 0.47 | 0.69 | 0.20 | 0.51 |
| | (0.32-0.53) | (0.34-0.59) | (0.58-0.79) | (0.12-0.30) | (0.40-0.62) |
| Specificity | 0.85 | 0.88 | 0.74 | 0.98 | 0.87 |
| | (0.80-0.89) | (0.84-0.92) | (0.69-0.80) | (0.97-1) | (0.83-0.91) |
| PPV | 0.47 | 0.56 | 0.46 | 0.81 | 0.56 |
| | (0.35-0.59) | (0.44-0.67) | (0.37-0.55) | (0.63-0.95) | (0.45-0.67) |
| NPV | 0.52 | 0.84 | 0.88 | 0.80 | 0.85 |
| | (0.78-0.86) | (0.80-0.88) | (0.84-0.92) | (0.75-0.84) | (0.81-0.89) |
| Accuracy | 0.75 | 0.78 | 0.73 | 0.80 | 0.79 |
| Balanced | 0.64 | 0.68 | 0.72 | 0.59 | 0.69 |
| accuracy | | | | | |

In Table 3, performance metrics (with 95% CI) of the individual gene expression profile and donor derived cfDNA assays are shown: (a) in combination with EITHER OR both tests being positive to diagnose subclinical rejection; (b) in combination with BOTH tests required to be positive to diagnose subclinical rejection, and (c) in the last column the performance of the multivariable logistic regression model combining the two assays using their continuous output scores. In Table 3 PPV denotes positive predictive value and NPV denotes negative predictive value.

Of true positive gene expression profile samples (n=44), 66% (n=29) of subclinical rejection cases detected were either acute cellular rejection or acute cellular rejection with antibody mediated rejection. The remaining 34% (n=15) were antibody mediated rejection alone. The timing post-transplant and type of rejection episodes detected by the gene expression profile is presented in Table 2.

Diagnostic Performance of Gene Expression, Donor Derived cfDNA, and the Combination of the Two Tests—Donor Derived cfDNA Performance

Of the 103 subclinical rejection cases, the donor derived cfDNA assay was positive in 47% (n=48) and negative in 53% (n=55). Of the 325 normal biopsy cases, the donor derived cfDNA assay was negative in 88% (n=287) and positive in 12% (n=38). Full performance metrics are listed in Table 3. Of true positive donor derived cfDNA samples (n=48), 92% (n=44) of subclinical rejection cases detected were either antibody mediated rejection or acute cellular rejection mixed with antibody mediated rejection (Table 2). In terms of timing, only 12.5% (n=6) of the true positive donor derived cfDNA results occurred before the 1-year post transplant biopsy, and all were antibody mediated rejection or acute cellular rejection mixed with antibody mediated rejection (Table 2).

When a donor derived cfDNA threshold of >1% was used as a positive cut-off, sensitivity (41%) was lower than using the 0.7% threshold (47%). However, specificity, PPV and NPV, accuracy, and balanced accuracy were similar or higher at 91%, 60%, 83%, 79%, and 66%, respectively (Table 4).

TABLE 4
Diagnostic performance of the donor derived cfDNA assay
depending on variable donor-derived cfDNA cut-off levels

| dd-cfDNA | | | | | | Balanced |
|---|---|---|---|---|---|---|
| cut-off | Sensitivity | Specificity | PPV | NPV | Accuracy | Accuracy |
| 0.35% | 0.69 | 0.66 | 0.39 | 0.87 | 0.66 | 0.68 |
| 0.5% | 0.52 | 0.78 | 0.43 | 0.84 | 0.72 | 0.65 |
| 0.7%* | 0.47 | 0.88 | 0.56 | 0.84 | 0.78 | 0.68 |
| 1% | 0.41 | 0.91 | 0.60 | 0.83 | 0.79 | 0.66 |

In Table 4, diagnostic performance characteristics of the donor derived cfDNA assay to differentiate subclinical acute rejection (subAR) from no rejection phenotypes based on a range of thresholds is depicted. In Table 4, the asterisk denotes the Viracor TRAC® assay cutoff threshold, NPV denotes negative predictive value, and PPV denotes positive predictive value.

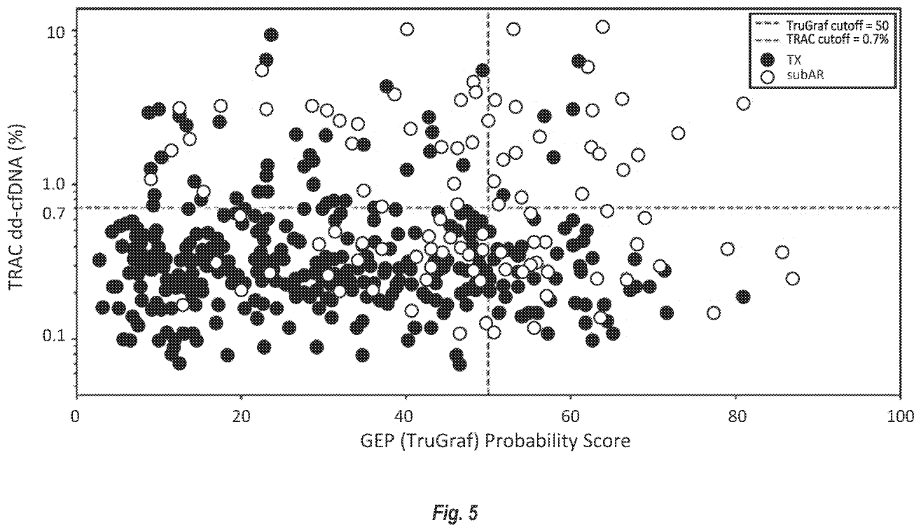

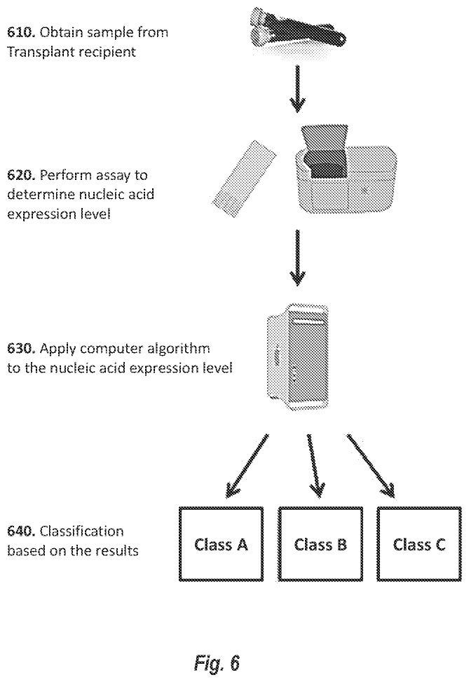

A scatterplot of all samples based on gene expression profile and donor derived cfDNA scores to present the distribution of scores for each assay is provided in FIG. 5. In FIG. 5, GEP denotes gene expression profile.

Performance of Gene Expression and Donor Derived cfDNA Depending on Rejection Type

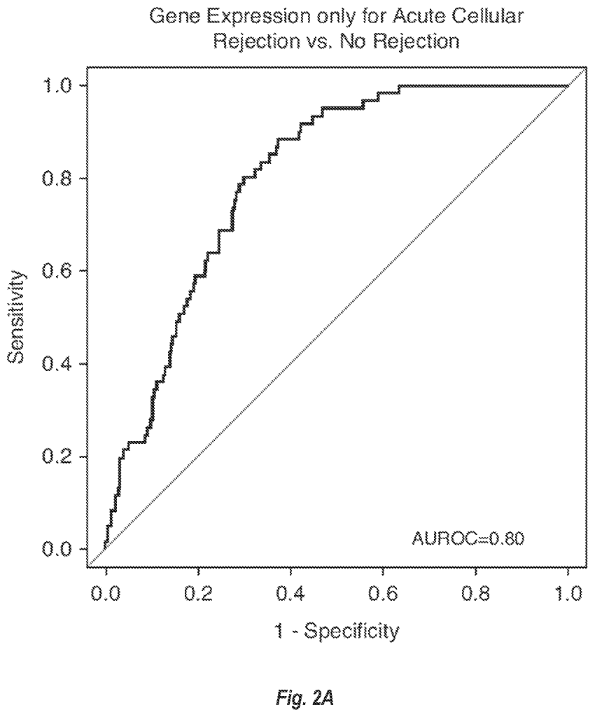

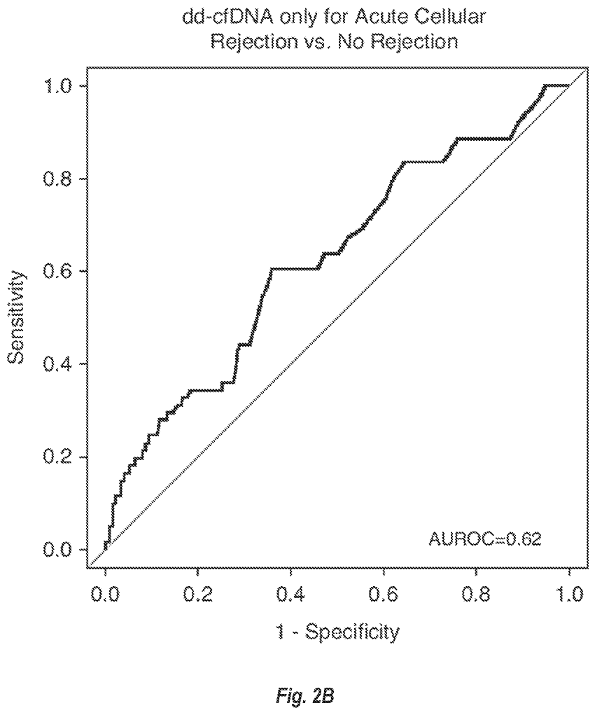

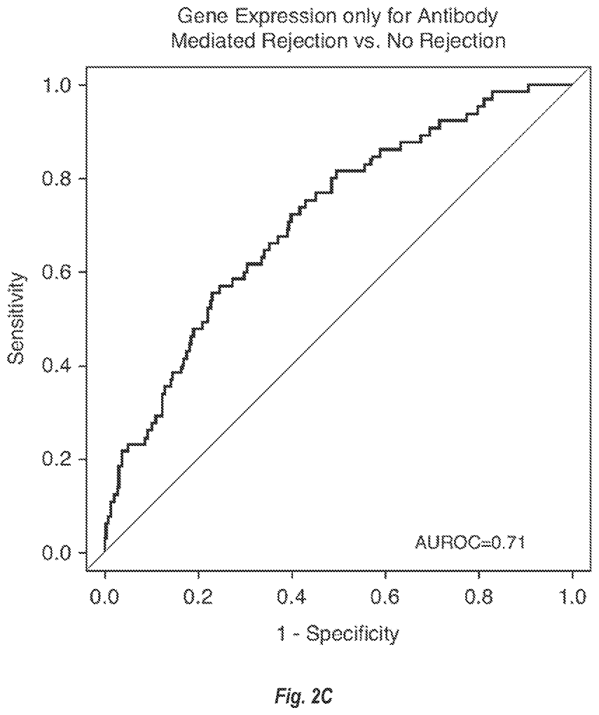

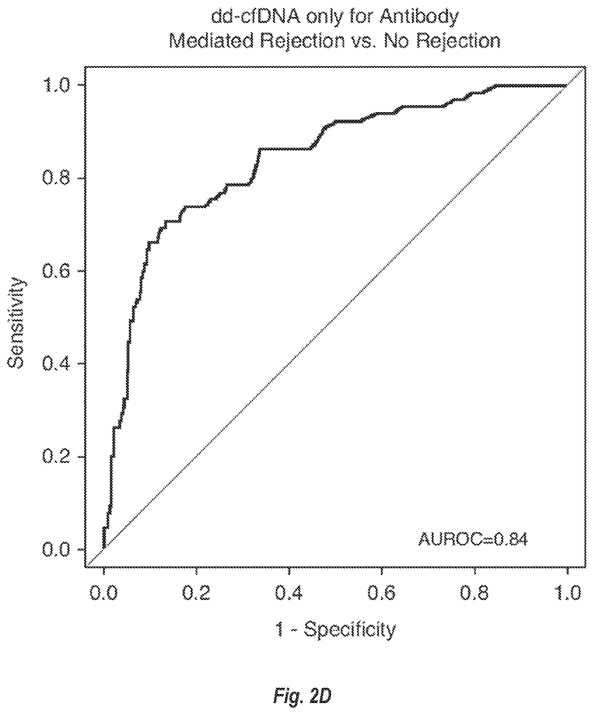

Significant differences were seen in diagnostic performance based on rejection type. The gene expression profile outperformed donor derived cfDNA on acute cellular rejection cases (that included acute cellular rejection alone and acute cellular rejection plus antibody mediated rejection cases) based on AUROC (0.80 vs 0.62, P<0.001) and balanced accuracy (0.67 vs 0.58, p=0.096) (FIG. 2 panel A and panel B). Conversely, donor derived cfDNA showed higher performance when compared to the gene expression profile in the antibody mediated rejection cases (that included antibody mediated rejection alone and antibody mediated rejection plus acute cellular rejection cases) based on AUROC (donor derived cfDNA 0.84 vs gene expression profile 0.71, P=0.003), and balanced accuracy (0.78 vs 0.62, p<0.001) (FIG. 2 panel C and panel D).

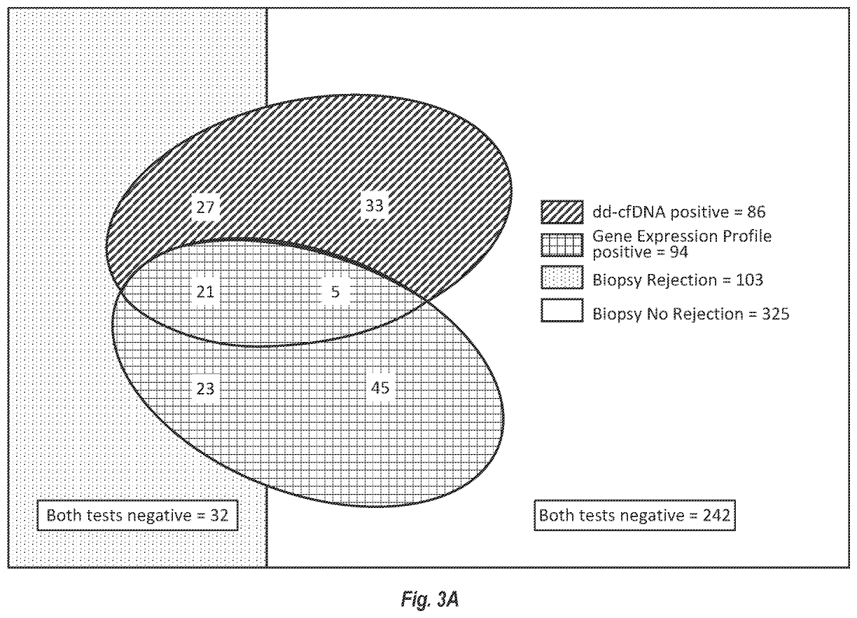

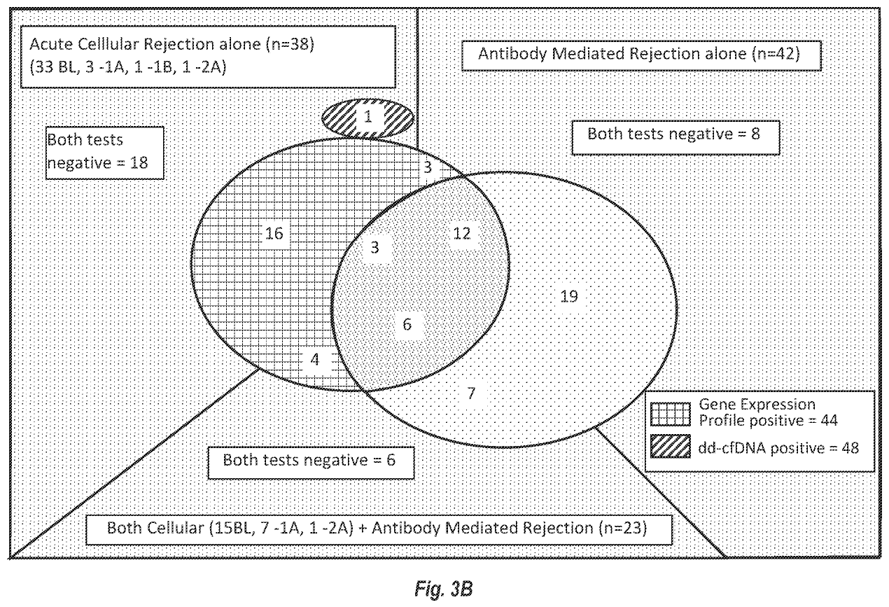

The overall summary of gene expression profile and donor derived cfDNA performance based on biopsy phenotype is summarized in (FIG. 3 panel A). Importantly, the figure highlights the overlap (or lack thereof) in which cases of subclinical rejection are accurately identified by the different tests, with the gene expression profile picking up more cases of earlier acute cellular rejection and donor derived cfDNA more cases of antibody mediated rejection (FIG. 3 panel B and Table 2). In addition, there is non-overlap of the biopsy paired samples that were called falsely positive by each test. The summary of diagnostic metrics for each rejection type is shown in Tables 5, 6, 7, and 8

TABLE 5
Summary of test results on all CTOT 08 samples by
rejection type using Gene Expression Profile, donor
derived-cfDNA, and the logistic regression model

| | Acute | Antibody- | |
|---|---|---|---|
| | Cellular | Mediated | |
| | Rejection + | Rejection + | No |
| | Mixed | Mixed | Rejection |
| | (n = 61) | (n = 65) | (n = 325) |
| GEP negative | 32 | 40 | 275 |
| GEP positive | 29 | 25 | 50 |
| dd-cfDNA negative | 44 | 21 | 287 |
| dd-cfDNA positive | 17 | 44 | 38 |
| LR negative | 31 | 27 | 283 |
| LR positive | 30 | 38 | 42 |

Table 5 lists the test results for all samples in the study cohort. The first column lists the cases of biopsy proven acute cellular rejection (which includes acute cellular rejection alone+acute cellular rejection mixed with antibody-mediated rejection). The second column similarly shows the cases of biopsy proven antibody-mediated rejection (which includes pure antibody—mediated rejection plus antibody-mediated rejection mixed with acute cellular rejection). The third column are the biopsies with no rejection. Each row shows the samples identified as positive or negative based on the assay. In Table 5, GEP denotes Gene Expression Profile assay, dd-cfDNA denotes donor derived cell free DNA assay, and LR denotes logistic regression model.

TABLE 6
Prediction by rejection type using gene expression
profile, donor derived-cfDNA, and logistic regression
on 105 sample external validation set.

| | Acute | Antibody- | |
|---|---|---|---|
| | Cellular | Mediated | No |
| | Rejection | Rejection | Rejection |
| | (n = 22) | (n = 11) | (n = 76) |
| GEP negative | 7 | 6 | 60 |
| GEP positive | 15 | 5 | 16 |
| dd-cfDNA negative | 14 | 4 | 66 |
| dd-cfDNA positive | 8 | 7 | 10 |
| LR negative | 9 | 2 | 62 |
| LR positive | 13 | 9 | 14 |

Table 6 lists the test results for all samples in the external validation set. The first column lists the cases of biopsy proven acute cellular rejection (which includes acute cellular rejection alone+acute cellular rejection mixed with antibody-mediated rejection). The second column similarly shows the cases of biopsy proven antibody-mediated rejection (which includes pure antibody-mediated rejection+antibody-mediated rejection mixed with acute cellular rejection). The third column lists the biopsies with no rejection. Each row shows the samples identified as positive or negative based on the assay. In Table 6, GEP denotes gene expression profile assay, dd-cfDNA denotes donor derived cell free DNA assay, and LR denotes logistic regression model.

TABLE 7
Gene expression profile and donor derived-cfDNA performance
on the antibody mediated rejection (n = 65) and no rejection
biopsy (n = 325) groups.

| | Sensi- | | | | | | Balanced |
|---|---|---|---|---|---|---|---|
| | tivity | Specificity | PPV | NPV | AUROC | Accuracy | Accuracy |
| GEP | 0.38 | 0.85 | 0.33 | 0.87 | 0.71 | 0.77 | 0.62 |
| dd- | 0.68 | 0.88 | 0.54 | 0.93 | 0.84 | 0.85 | 0.78 |
| cfDNA | | | | | | | |

Table 7 lists the performance of the GEP and dd-cfDNA assays distinguishing the subset of antibody mediated rejection (AMR) and no rejection samples. In Table 7 GEP denotes gene expression profile assay, dd-cfDNA denotes donor derived cell free DNA assay, PPV denotes positive predictive value, NPV denotes negative predictive value, and AUROC denotes area under the receiver operator curve.

TABLE 8
Gene expression profile and donor derived-cfDNA performance on the
acute cellular rejection (n = 61) and no rejection (n = 325) groups.

| | Sensi- | | | | | | Balanced |
|---|---|---|---|---|---|---|---|
| | tivity | Specificity | PPV | NPV | AUROC | Accuracy | Accuracy |
| GEP | 0.48 | 0.85 | 0.37 | 0.90 | 0.80 | 0.79 | 0.67 |
| dd- | 0.28 | 0.88 | 0.31 | 0.87 | 0.62 | 0.79 | 0.58 |
| cfDNA | | | | | | | |

Table 8 lists the performance of the gene expression profile and donor derived-cfDNA assays distinguishing the subset of acute cellular rejection and no rejection samples. In Table 8, GEP denotes gene expression profile assay, dd-cfDNA denotes donor derived cell free DNA assay, PPV denotes positive predictive value, NPV denotes negative predictive value, and AUROC denotes area under the receiver operator curve.

Additionally, during the current study, previous summary of biopsy phenotypes by the study participants were updated to the Banff 2019 classification (see e.g. Loupy et al. Am J Transplant. 2020 September; 20(9):2318-2331, which is incorporated by reference in its entirety herein). Table 9 depicts changes in categorization according to the newer Banff 2019 classification.

TABLE 9
Summary of biopsy phenotypes based on the prior Banff
grading and the updated Banff 2019 classification

| Original | Reclassification | Summary of the |
|---|---|---|
| pathology | results with | changes in Banff |
| grading | Banff 2019 | scoring at the sample |
| (n = 428) | (n = 428) | level |
| Rejection (n = 104) | Rejection (n = 83) | Rejection (n = 103) |
| Borderline (n = 72) | Borderline (n = 33) | Borderline |
| −i0t1 (n = 21) | ≥1A (n = 5), | (n = 33) |
| −i0t2 (n = 20) | 1A + suspicious AMR | ≥1A (n = 5) |
| −i0t3 (n = 3) | (n = 4), | 1A + suspicious |
| −i0t0 (n = 2) | AMR (n = 15), | AMR (n = 4) |
| −i1t0 (n = 1) | AMR + borderline (n = 2), | AMR (n = 20) |
| −i1t1, t2 or 13 (n = 18) | AMR + ≥1A (n = 4) | AMR + |
| −i2t1 (n = 1) | Borderline + suspicious | borderline |
| −i2t2 (n = 2) | AMR (n = 13) | (n = 2) |
| −i3t1 (n = 2) | Suspicious AMR (n = 7) | AMR + ≥1A |
| −i3t2(n = 1) | No Rejection (TX) (n = 21) | (n = 4) |
| −i3t3 (n = 1) | l0t1 (n = 18) | Borderline + |
| 1A (n = 5) | l0t0 (n = 2), 1110 (n = 1) | suspicious |
| AMR (n = 19) | | AMR (n = 13) |
| AMR + ≥1A (n = 3) | | Suspicious AMR |
| AMR + borderline | | (n = 22) |
| (n = 5) | | |
| No Rejection (n = 324) | Rejection (n = 20) | No Rejection (n = 325) |
| | Suspicious AMR (n = 15) | |
| | AMR (n = 5) | |
| | No Rejection (n = 304) | |

In Table 9, changes in participant classification according to the Banff 2019 scheme are shown. Of 21 cases of i0t1, 18 and 3 cases were reclassified to the no rejection and suspicious AMR group, respectively. The cases with i0t2 (n=20) and i0t3 (n=3) remained in the Rejection group.

Combined Gene Expression Profile and Donor Derived cfDNA Performance

The diagnostic performance was investigated relative to biopsy phenotypes with different combination groups of gene expression profile and donor derived cfDNA tests. Of time points with subclinical rejection on biopsy and both gene expression profile and donor derived cfDNA positive (n=21), 12 were antibody mediated rejection alone, 3 acute cellular rejection alone, and 6 combined antibody mediated rejection+acute cellular rejection by histology (FIG. 3 panel A and panel B). Thirty-two subclinical rejection cases found on biopsy were both gene expression profile and donor derived cfDNA negative (FIG. 3 panel A). The histology of those associated samples was borderline (n=15), Banff 1A (n=3), Banff 1A with antibody mediated rejection including suspicious antibody mediated rejection (n=2), borderline with suspicious antibody mediated rejection (n=4), suspicious antibody mediated rejection (n=7), and antibody mediated rejection (n=1).

First, the group with either gene expression profile or donor derived cfDNA positive including both tests positive (Positive=gene expression profile OR donor derived cfDNA positive) was considered as a positive test and compared to those where both gene expression profile and donor derived cfDNA were negative to be called negative (Table 3). Using this combination as the definition of positive and negative, the NPV increased to 88% (95% CI, 0.84-0.92). When requiring both gene expression profile and donor derived cfDNA to be positive (Positive=gene expression profile AND donor derived cfDNA positive) to call a time point a positive test, the sensitivity dropped to 20% (95% CI, 0.12-0.30), but the specificity increased to 98% (95% CI, 0.97-1) with a PPV of 0.81 (95% CI, 0.63-0.95). A summary of the performance characteristics is presented in Table 3.

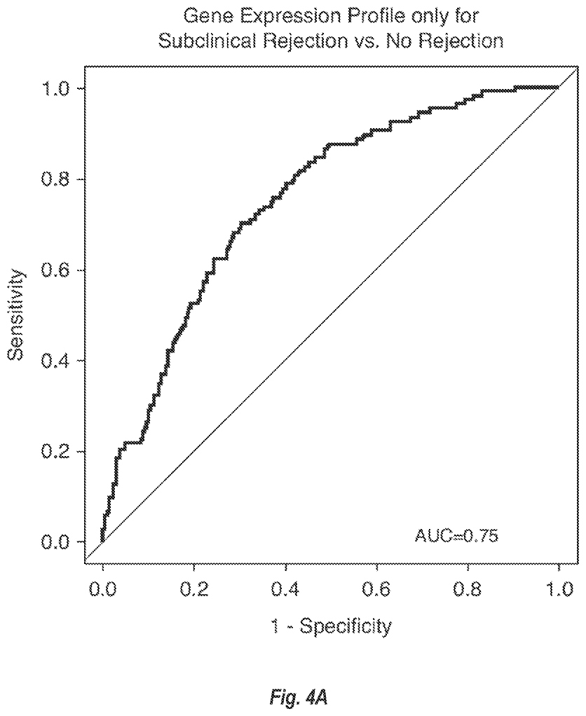

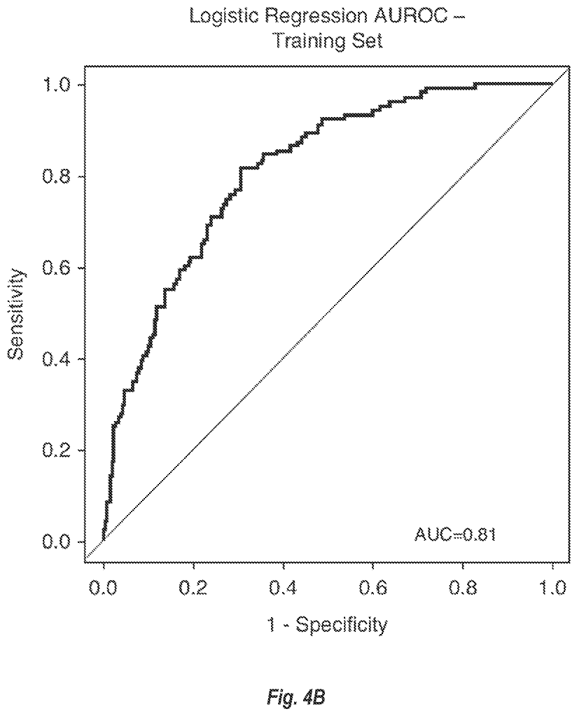

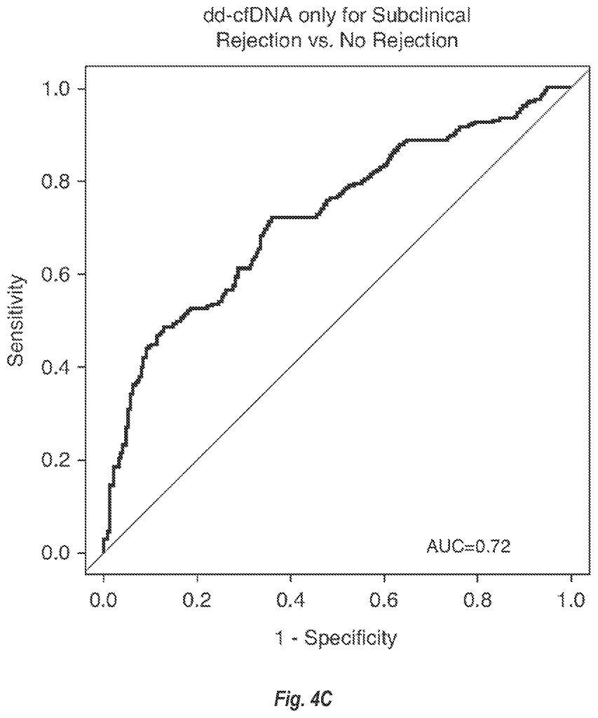

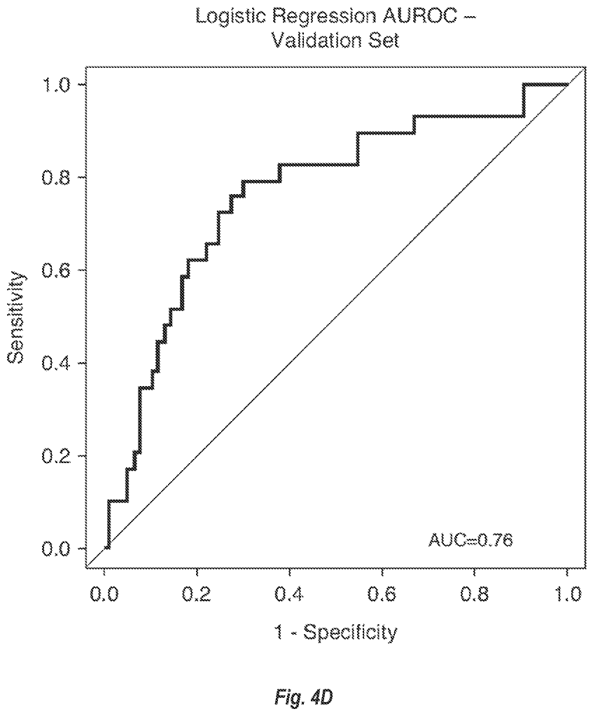

When the gene expression profile and donor derived cfDNA were combined using a multivariable logistic regression using their continuous scores rather than binary output based on thresholds, the AUROC improved to 0.81, which was significantly higher than gene expression profile alone (0.81 vs. 0.75, P<0.001) or donor derived cfDNA alone (0.81 vs. 0.72, P=0.006) (FIG. 4 panels A, B, and C). Examining the continuous scores, a 1% higher donor derived cfDNA is associated with an odds ratio of 1.76 (95% CI, 1.38-2.23, P<0.001) for subclinical rejection, and each 10-point higher gene expression profile probability score is associated with an odds ratio of 1.66 (95% CI, 1.43-1.92, P<0.001) for subclinical rejection. This suggests that the donor derived cfDNA and gene expression profile assays are independently associated with subclinical rejection.

### Logistic Regression Model External Validation

The external validation AUROC (0.76) for the combined test logistic regression model maintained good performance compared with the CTOT-08 dataset (0.81) (FIG. 4 panel D). The prediction by rejection type using gene expression profile, donor derived cfDNA, and logistic regression on the CTOT-08 and external validation set is available as supplemental Tables 3 and 4.

### D. Discussion

In the study described herein, the diagnostic performance of a blood gene expression profile biomarker and plasma donor derived cfDNA assay in stable kidney transplant patients with either normal surveillance biopsies or subclinical rejection was described. Importantly, this is believed to be the first study to define the performance characteristics of donor derived cfDNA in a large, prevalent cohort of stable patients that all had surveillance kidney biopsies. With recent updates to the Banff classification (see e.g. Loupy et al. Am J Transplant. 2020 September; 20(9):2318-2331, which is incorporated by reference in its entirety herein), the bar was raised for the diagnosis of cellular rejection (with the elimination of the i0, t1 lesions in the borderline category). Conversely, parameters were chosen to be more inclusive for diagnosing antibody mediated rejection (by including cases in the suspicious antibody mediated rejection category which we and others believe represents consequential microvascular inflammation). The net effect was to increase the number of cases of subclinical antibody mediated rejection and reduce the number of subclinical acute cellular rejection cases seen in our observational cohort.

This series establishes the performance of donor derived cfDNA as a screening test in a prevalent cohort of stable kidney recipients. In addition, this is believed to be the first report to characterize the combined performance of donor derived cfDNA and a gene expression profile test at the same time points, with all clinical phenotypes confirmed by surveillance biopsy. While the two tests have similar overall sensitivity and specificity, the gene expression profile preferentially detects subclinical acute cellular rejection while donor derived cfDNA preferentially detects subclinical antibody mediated rejection (see e.g. Tables 5, 6, 7, and 8). In the observational trial, subclinical acute cellular rejection tended to occur earlier in the two-year follow up than antibody mediated rejection (except for patients that underwent positive crossmatch desensitization). This has important implications for their use in clinical practice, in that detecting early cellular rejection using the gene expression profile may prevent antibody formation later post-transplant.

The data also indicate the complementarity of donor derived cfDNA and gene expression assays. Although both assays have similar rates of false positives (15% for gene expression profile and 12% for donor derived cfDNA, respectively), they call a different set of samples falsely positive (FIG. 3A). Similarly, there is a fair amount of non-overlap in true positives called by each test (FIG. 3A-3B). Accordingly, the performance metrics improved when both gene expression profile and donor derived cfDNA results were considered together, primarily based on the detection of specific histological subtypes. Further implementation of the assays as a graded output may further improve detection along these lines as there is a graded risk of rejection as the value of the continuous output of both assays increases.

The current study has multiple strengths including 1) gene expression profile and donor derived cfDNA were evaluated simultaneously with each biopsy. 2) the clinical trial used strictly defined and objective clinical criteria for subclinical rejection. 3) the study population was a representative US kidney transplant population, 4) biopsies were read by a central pathologist to reduce interobserver variability.

Blood-based biomarkers such as described herein may allow less invasive, more frequent monitoring of kidney transplant recipients for subclinical rejection. Donor derived cfDNA was significantly better at detecting subclinical antibody mediated rejection when compared with the gene expression profile, and conversely the gene expression profile was significantly better at detecting subclinical acute cellular rejection. When both gene expression profile and donor derived cfDNA are negative or positive, their NPV or PPV is higher than either test alone. Combining the continuous output scores of both tests using a novel multivariable logistic regression model significantly improved the AUROC when compared to either test alone.

### Example 2. Gene Expression Profile Implemented as qPCR Assay

Performance of the combined dd-cfDNA and mRNA expression analysis methods was determined when comparing mRNA expression data obtained by microarray to that obtained by quantitative PCR. Using the same biopsy-paired samples as in Example 1, there was an improvement in the NPV from 88% to 94% when both TRAC®; dd-cfDNA and TruGraf® assays were negative, and an increase in PPV from 81% to 89% when both were positive. False negative results were reduced from 31% to 17%, while true negative results improved from 74% to 81%. Within the cohorts, 26.2% of results were positive for one test and negative for the other (11.7% TRAC+ and TruGraf−: 14.5% TRAC− and TruGraf+). The methodological improvement in TruGraf® technology increased its detection of both acute cellular rejection (T cell mediated rejection) and antibody mediated rejection subtypes, leading to a higher NPV and PPV.

As in Example 1, dd-cfDNA (TRAC′R assay) results are considered positive (or non-TX) when dd-cfDNA is ≥ 0.7% and negative (TX) when dd-cfDNA is <0.7%. For the TruGraf® gene expression assay, reverse transcriptase polymerase chain reaction (RT-PCR) and microfluidics on the Fluidigm Biomark HD™ System (Fluidigm, South San Francisco, CA) was used to provide rapid quantitative analysis of mRNA expression, while requiring less RNA input and reducing turnaround time compared to microarray processes such as used in Example 1. Furthermore, sample volume for the assays could be reduced to 6 mL of blood from 15 mL of blood used for microarray methods.

In general, more positive results were found by PCR than by microarray. Out of the original 428 samples, 103 had a biopsy result indicating subclinical acute rejection. Of these, 36 were found to be positive only in the gene expression assay (vs 23 by methods of Example 1), 32 were found positive in both assays (vs 21 by methods of Example 1), 17 by only the dd-cfDNA assay (vs 27 by methods of Example 1), and 18 were negative in both tests (vs 32 in methods of Example 1). Table 10 below provides the performance metrics (Cl=confidence limit; PPV=positive predictive value; NPV=negative predictive value).

TABLE 10
Performance metrics for dd-cfDNA and quantitative
PCR gene expression assay

| | | | Positive = | Positive = |
|---|---|---|---|---|
| | TruGraf ® | Viracor | Either | Both |
| | assay | TRAC ® | TruGraf ® | TruGraf ® |
| Diagnostic | alone | assay | or TRAC ® | AND TRAC ® |
| Performance | (95% CI) | (95% CI) | assay | assays |
| Sensitivity | 0.72 | 0.47 | 0.69 | 0.77 |
| | (0.68-0.83) | (0.34-0.59) | (0.58-0.79) | (0.71-0.80) |
| Specificity | 0.85 | 0.88 | 0.74 | 0.94 |
| | (0.80-0.89) | (0.84-0.92) | (0.69-0.80) | (0.92-1) |
| PPV | 0.65 | 0.56 | 0.46 | 0.89 |
| | (0.61-0.70) | (0.44-0.67) | (0.37-0.55) | (0.84-0.95) |
| NPV | 0.91 | 0.84 | 0.94 | 0.81 |
| | (0.86-0.94) | (0.80-0.88) | (0.92-1) | (0.63-0.95) |
| Accuracy | 0.75 | 0.78 | 0.73 | 0.85 |
| False | 8% | 12% | 6% | 4.5% |
| Positive Rate | | | | |

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of distinguishing rejection from non-rejection in a kidney transplant recipient, the method comprising
a. obtaining a blood, plasma, or serum sample from the kidney transplant recipient;
b. obtaining cell-free DNA (cfDNA) and mRNA from the sample;
c. determining (i) the level of donor derived cell-free DNA (dd-cfDNA) in the cfDNA and (ii) the expression level of at least one mRNA transcript, wherein the at least one mRNA transcript shows significantly different expression levels in kidney transplant rejection compared to kidney transplant non-rejection subjects; and
d. distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) result of a trained algorithm based on the expression level of the at least one mRNA transcript indicating rejection or non-rejection, wherein the algorithm compares the expression profile of the at least one mRNA transcript of the recipient to the expression profile of kidney transplant subjects with and without rejection.

2. The method of claim 1, wherein rejection in the recipient is indicated by a pre-determined threshold value of dd-cfDNA of ≥0.5%, ≥0.6%, ≥0.7%, ≥0.8%, ≥0.9%, ≥1%, ≥1.2%, ≥1.5%, or ≥2%.

3. The method of claim 2, wherein rejection in the recipient is indicated by a pre-determined threshold value of dd-cfDNA of ≥0.7%, optionally wherein determining the dd-cfDNA level utilizes data from recipient genotype information.

4. The method of any one of claims 1-3, wherein the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample.

5. The method of claim 4, wherein the at least one mRNA transcript comprises one or more of the mRNA transcripts of Table A.

6. The method of claim 5, wherein the at least one mRNA transcript comprises 2-120, 5-120, 10-120, 50-120, 80-120, 2-128, 5-128, 10-128, 50-128, 80-128, 5-50, 10-50, 50-100, or all of the mRNA transcripts of Table A.

7. The method of any one of claims 1-6, wherein the recipient has a serum creatinine level of <2.3 mg/dL, or an increase of serum creatinine compared to baseline of no more than 10% or no more than 20%.

8. The method of any one of claims 1-6, wherein the recipient has a serum creatinine level of 2.3 mg/dL or higher, or an increase of serum creatinine compared to baseline of no more than 10% or no more than 20%.

9. The method of any one of claims 1-8, wherein the method is performed at least one month, at least two months, at least three months, at least six months, or at least one year after transplantation.

10. The method of any one of claims 1-9, wherein the expression level of the at least one mRNA transcript is determined by reverse transcription PCR (RT-PCR) (such as quantitative RT-PCR), hybridization to an array, or next generation sequencing.

11. The method of any one of claims 1-10, wherein the dd-cfDNA level is determined by whole genome sequencing.

12. The method of any one of claims 1-11, wherein determining the dd-cfDNA level comprises comparison of recipient and donor genotype information.

13. The method of any one of claims 1-11, wherein the dd-cfDNA is determined without comparison to donor genotype information.

14. The method of any one of claims 1-13, wherein the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects.

15. The method of any one of claims 1-14, wherein the method is capable of further distinguishing likelihood of acute cellular rejection from antibody-mediated rejection, wherein the dd-cfDNA level indicates presence or absence of antibody-mediated rejection, and wherein the level of the at least one mRNA transcript indicates presence or absence of acute cellular rejection.

16. The method of any one of claims 1-15, wherein the method has a negative predictive value (NPV) of at least 85%, at least 87%, at least 88%, at least 90%, at least 92%, or at least 94% when both the level of dd-cfDNA is below the pre-determined threshold value and the result of a trained algorithm based on the expression level of the at least one mRNA transcript does not indicate rejection.

17. The method of any one of claims 1-16, wherein the method has a positive predictive value (NPV) of at least 80%, at least 81%, at least 82%, at least 84%, at least 86%, at least 88%, or at least 89% when both the level of dd-cfDNA is at or above the pre-determined threshold value and the result of a trained algorithm based on the expression level of the at least one mRNA transcript indicates rejection.

18. The method of claim 16 or 17, wherein determining the dd-cfDNA level utilizes data from recipient genotype information and wherein the expression level of the at least one mRNA transcript is determined by reverse-transcription PCR (RT-PCR) (such as quantitative RT-PCR).

19. The method of any one of claims 1-18, wherein the pre-determined threshold value of the dd-cfDNA is determined by a multivariate regression algorithm that comprises dd-cfDNA levels and expression levels of the at least one mRNA transcript in a set of transplant recipients who received the same solid organ transplant as the recipient.
